SEQUENCE LISTING (40 sequences)

SEQ ID NO: 1 - DNA, 1317 bp, Escherichia coli
  CDS (1)..(1317)
atgggaaacacatcaatacaaacgcagagttaccgtgcggtagataaagatgcagggcaaagcagaagttacattattccattcgcgctgctgtgctcactgttttttctttgggcggtagccaataaccttaacgacattttattacctcaattccagcaggcttttacgctgacaaatttccaggctggcctgatccaatcggccttttactttggttatttcattatcccaatccctgctgggatattgatgaaaaaactcagttataaagcagggattattaccgggttatttttatatgccttgggtgctgcattattctggcccgccgcagaaataatgaactacaccttgtttttagttggcctatttattattgcagccggattaggttgtctggaaactgccgcaaacccttttgttacggtattagggccggaaagtagtggtcacttccgcttaaatcttgcgcaaacatttaactcgtttggcgcaattatcgcggttgtctttgggcaaagtcttattttgtctaacgtgccacatcaatcgcaagacgttctcgataaaatgtctccagagcaattgagtgcgtataaacacagcctggtattatcggtacagacaccttatatgatcatcgtggctatcgtgttactggtcgccctgctgatcatgctgacgaaattcccggcattgcagagtgataatcacagtgacgccaaacaaggatcgttctccgcatcgctttctcgcctggcgcgtattcgccactggcgctgggcggtattagcgcaattctgctatgtcggcgcacaaacggcctgctggagctatttgattcgctacgctgtagaagaaattccaggtatgactgcaggctttgccgctaactatttaaccggaaccatggtgtgcttctttattggtcgtttcaccggtacctggctcatcagtcgcttcgcaccacacaaagtcctggccgcctacgcattaatcgctatggcactgtgcctgatctcagccttcgctggcggtcatgtgggcttaatagccctgactttatgcagcgcctttatgtcgattcagtacccaacaatcttctcgctgggcattaagaatctcggccaggacaccaaatatggttcgtccttcatcgttatgaccattattggcggcggtattgtcactccggtcatgggttttgtcagtgacgcggcgggcaacatccccactgctgaactgatccccgcactctgcttcgcggtcatctttatctttgcccgtttccgttctcaaacggcaactaactga

SEQ ID NO: 2 - protein, 438 aa, Escherichia coli
MGNTSIQTQSYRAVDKDAGQSRSYIIPFALLCSLFFLWAVANNLNDILLPQFQQAFTLTNFQAGLIQSAFYFGYFIIPIPAGILMKKLSYKAGIITGLFLYALGAALFWPAAEIMNYTLFLVGLFIIAAGLGCLETAANPFVTVLGPESSGHFRLNLAQTFNSFGAIIAVVFGQSLILSNVPHQSQDVLDKMSPEQLSAYKHSLVLSVQTPYMIIVAIVLLVALLIMLTKFPALQSDNHSDAKQGSFSASLSRLARIRHWRWAVLAQFCYVGAQTACWSYLIRYAVEEIPGMTAGFAANYLTGTMVCFFIGRFTGTWLISRFAPHKVLAAYALIAMALCLISAFAGGHVGLIALTLCSAFMSIQYPTIFSLGIKNLGQDTKYGSSFIVMTIIGGGIVTPVMGFVSDAAGNIPTAELIPALCFAVIFIFARFRSQTATN

SEQ ID NO: 3 - DNA, 1776 bp, Escherichia coli
  CDS (1)..(1776)
atgaaaaaaatcagcttaccgaaaattggtatccgcccggttattgacggtcgtcgcatgggtgttcgtgagtcgcttgaagaacaaacaatgaatatggcgaaagctacggccgcactgctgaccgagaaactgcgccatgcctgcggagctgccgtcgagtgtgtcatttccgatacctgtatcgcgggtatggctgaagccgctgcttgcgaagaaaaattcagcagtcagaatgtaggcctcaccattacggtaacgccttgctggtgctatggcagtgaaaccatcgacatggatccaacccgcccgaaggccatttggggctttaacggcactgaacgccccggcgctgtttacctggcagcggctctggcagctcacagccagaaaggcatcccagcattctccatttacggtcatgacgttcaggatgccgatgacacatcgattcctgccgatgttgaagaaaaactgctgcgctttgcccgcgccggtttggccgtcgccagcatgaaaggtaaaagctatctgtcgctgggcggcgtttcgatgggtatcgccggttccattgttgatcacaacttctttgaatcctggctgggaatgaaagtccaggcggtggatatgaccgaactgcgtcgccgtatcgatcagaagatttacgacgaagccgaattggaaatggcactggcctgggctgataaaaacttccgctatggcgaagatgaaaataacaaacagtatcaacgtaatgccgagcaaagccgcgcagttctgcgcgaaagtttactgatggcgatgtgtatccgcgacatgatgcaaggcaacagcaaactggccgatattggtcgcgtggaagaatcacttggctacaacgccatcgctgcgggcttccaggggcaacgtcactggaccgatcaatatcccaatggtgacaccgccgaagcgatcctcaacagttcatttgactggaatggcgtgcgcgaaccctttgtcgtggcgaccgaaaacgacagtcttaacggcgtggcaatgctaatgggtcaccagctcaccggcaccgctcaggtatttgccgatgtgcgtacctactggtcaccagaagcaattgagcgtgtaacggggcataaactggatggactggcagaacacggcatcatccatttgatcaactccggttctgctgcgctggacggttcctgtaaacaacgcgacagcgaaggtaacccgacgatgaagccacactgggaaatctctcagcaagaggctgacgcttgcctcgccgctaccgaatggtgcccggcgatccacgaatacttccgtggcggcggttactcttcccgcttccttaccgaaggcggcgtcccgttcaccatgactcgtgtcaacatcatcaaaggcctgggaccggtactgcaaatcgcggaaggctggagcgtggaattgccgaaggatgtgcatgacatcctcaacaaacgcaccaactcaacctggccaaccacctggtttgcaccgcgcctcaccggtaaagggccgtttacggatgtgtactcggtaatggcgaactggggcgctaaccatggggttctgaccatcggccacgttggcgcagactttatcactctcgcctccatgctgcgtatcccggtatgtatgcacaacgttgaagagaccaaagtgtatcgtccttctgcctgggctgcgcacggcatggatattgaaggccaggattaccgcgcttgccagaactacggtccgttgtacaagcgttaa

SEQ ID NO: 4 - protein, 591 aa, Escherichia coli
MKKISLPKIGIRPVIDGRRMGVRESLEEQTMNMAKATAALLTEKLRHACGAAVECVISDTCIAGMAEAAACEEKFSSQNVGLTITVTPCWCYGSETIDMDPTRPKAIWGFNGTERPGAVYLAAALAAHSQKGIPAFSIYGHDVQDADDTSIPADVEEKLLRFARAGLAVASMKGKSYLSLGGVSMGIAGSIVDHNFFESWLGMKVQAVDMTELRRRIDQKIYDEAELEMALAWADKNFRYGEDENNKQYQRNAEQSRAVLRESLLMAMCIRDMMQGNSKLADIGRVEESLGYNAIAAGFQGQRHWTDQYPNGDTAEAILNSSFDWNGVREPFVVATENDSLNGVAMLMGHQLTGTAQVFADVRTYWSPEAIERVTGHKLDGLAEHGIIHLINSGSAALDGSCKQRDSEGNPTMKPHWEISQQEADACLAATEWCPAIHEYFRGGGYSSRFLTEGGVPFTMTRVNIIKGLGPVLQIAEGWSVELPKDVHDILNKRTNSTWPTTWFAPRLTGKGPFTDVYSVMANWGANHGVLTIGHVGADFITLASMLRIPVCMHNVEETKVYRPSAWAAHGMDIEGQDYRACQNYGPLYKR

SEQ ID NO: 5 - DNA, 1449 bp, Escherichia coli
  CDS (1)..(1449)
atgttatccggctatattgcaggagcgattatgaaacaagaagttatcctggtactcgactgtggcgcgaccaatgtcagggccatcgcggttaatcggcagggcaaaattgttgcccgcgcctcaacgcctaatgccagcgatatcgcgatggaaaacaacacctggcaccagtggtctttagacgccattttgcaacgctttgctgattgctgtcggcaaatcaatagtgaactgactgaatgccacatccgcggtatcgccgtcaccacctttggtgtggatggcgctctggtagataagcaaggcaatctgctctatccgattattagctggaaatgtccgcgaacagcagcggttatggacaatattgaacggttaatctccgcacagcggttgcaggctatttctggcgtcggagcctttagtttcaatacgttatataagttggtgtggttgaaagaaaatcatccacaactgctggaacgcgcgcacgcctggctctttatttcgtcgctgattaaccaccgtttaaccggcgaattcactactgatatcacgatggccggaaccagccagatgctggatatccagcaacgcgatttcagtccgcaaattttacaagccaccggtattccacgccgactcttccctcgtctggtggaagcgggtgaacagattggtacgctacagaacagcgccgcagcaatgctcggcttacccgttggcataccggtgatttccgcaggtcacgatacccagttcgccctttttggcgctggtgctgaacaaaatgaacccgtgctctcttccggtacatgggaaattttaatggttcgcagcgcccaggttgatacttcgctgttaagtcagtacgccggttccacctgcgaactggatagccaggcagggttgtataacccaggtatgcaatggctggcatccggcgtgctggaatgggtgagaaaactgttctggacggctgaaacaccctggcaaatgttgattgaagaagctcgtctgatcgcgcctggcgcggatggcgtaaaaatgcagtgtgatttattgtcgtgtcagaacgctggctggcaaggagtgacgcttaataccacgcgggggcatttctatcgcgcggcgctggaagggttaactgcgcaattacagcgcaatctacagatgctggaaaaaatcgggcactttaaggcctctgaattattgttagtcggtggaggaagtcgcaacacattgtggaatcagattaaagccaatatgcttgatattccggtaaaagttctcgacgacgccgaaacgaccgtcgcaggagctgcgctgttcggttggtatggcgtaggggaatttaacagcccggaagaagcccgcgcacagattcattatcagtaccgttatttctacccgcaaactgaacctgaatttatagaggaagtgtga

SEQ ID NO: 6 - protein, 482 aa, Escherichia coli
MLSGYIAGAIMKQEVILVLDCGATNVRAIAVNRQGKIVARASTPNASDIAMENNTWHQWSLDAILQRFADCCRQINSELTECHIRGIAVTTFGVDGALVDKQGNLLYPIISWKCPRTAAVMDNIERLISAQRLQAISGVGAFSFNTLYKLVWLKENHPQLLERAHAWLFISSLINHRLTGEFTTDITMAGTSQMLDIQQRDFSPQILQATGIPRRLFPRLVEAGEQIGTLQNSAAAMLGLPVGIPVISAGHDTQFALFGAGAEQNEPVLSSGTWEILMVRSAQVDTSLLSQYAGSTCELDSQAGLYNPGMQWLASGVLEWVRKLFWTAETPWQMLIEEARLIAPGADGVKMQCDLLSCQNAGWQGVTLNTTRGHFYRAALEGLTAQLQRNLQMLEKIGHFKASELLLVGGGSRNTLWNQIKANMLDIPVKVLDDAETTVAGAALFGWYGVGEFNSPEEARAQIHYQYRYFYPQTEPEFIEEV

SEQ ID NO: 7 - DNA, 423 bp, Escherichia coli
  CDS (1)..(423)
atgctgaaaacaatttcgccgttaatttctcccgaactattgaaagtgctggcagagatgggacatggagatgaaattattttttccgatgctcactttcccgcccattcgatgggaccgcaggtgatccgcgctgatggcctgttggtgagcgacttgctccaggcgattatcccgttatttgaactggacagttatgcaccgccgctggtgatgatggcggcggtagaaggtgacactctcgatcctgaagtagaacgacgttaccgtaatgcgctttcactacaagccccgtgtcctgacatcatccgcatcaatcgttttgcgttttatgaacgggcgcaaaaagcctttgcgatcgttatcacaggcgaacgagcgaagtacgggaatattcttttaaaaaaaggggtaacaccgtaa

SEQ ID NO: 8 - protein, 140 aa, Escherichia coli
MLKTISPLISPELLKVLAEMGHGDEIIFSDAHFPAHSMGPQVIRADGLLVSDLLQAIIPLFELDSYAPPLVMMAAVEGDTLDPEVERRYRNALSLQAPCPDIIRINRFAFYERAQKAFAIVITGERAKYGNILLKKGVTP

SEQ ID NO: 9 - DNA, 732 bp, Escherichia coli
  CDS (1)..(732)
atgaaagcggcacgccagcaagcgatagtcgacctgctgctgaaccataccagcctgaccacggaagctctctctgaacagctaaaggtcagtaaagaaaccattcgtcgcgatctcaatgaattacagacgcagggtaaaattctgcgcaatcatggacgcgctaaatatatccaccgtcaaaatcaagacagtggcgatccctttcacatcaggctgaaaagccattatgcgcataaagcagatatcgcgcgcgaggcgctcgcgtggattgaagaagggatggtgatagccttagacgccagttcaacttgctggtatctggcacgccagttgcctgacatcaacattcaggtcttcaccaatagccatccgatttgccatgaactcggtaaacgcgaacgcattcaactgatcagttccggcggcacacttgagcgcaaatatggctgttacgtcaatccctcgctgatttcccaacttaaatcgctggaaatcgatctgtttattttttcttgtgaagggatcgatagcagcggcgcactgtgggactccaatgcgatcaacgctgattacaaatcgatgctattaaaacgtgccgcgcaatcgttgttattgattgataaaagtaaatttaatcgttcaggggaagcccgcatcgggcatctggatgaggtaacgcacattatttctgatgagcgccaggttgcaacttctttggtaacagcctga

SEQ ID NO: 10 - protein, 243 aa, Escherichia coli
MKAARQQAIVDLLLNHTSLTTEALSEQLKVSKETIRRDLNELQTQGKILRNHGRAKYIHRQNQDSGDPFHIRLKSHYAHKADIAREALAWIEEGMVIALDASSTCWYLARQLPDINIQVFTNSHPICHELGKRERIQLISSGGTLERKYGCYVNPSLISQLKSLEIDLFIFSCEGIDSSGALWDSNAINADYKSMLLKRAAQSLLLIDKSKFNRSGEARIGHLDEVTHIISDERQVATSLVTA

SEQ ID NO: 11 - DNA, 120 bp, Artificial
  DNA fragment containing attL
agatcttgaagcctgcttttttatactaagttggcattataaaaaagcattgcttatcaatttgttgcaacgaacaggtcactatcagtcaaaataaaatcattatttgatttcgaattc

SEQ ID NO: 12 - DNA, 40 bp, Artificial
  primer
ctagtaagatcttgaagcctgcttttttatactaagttgg

SEQ ID NO: 13 - DNA, 41 bp, Artificial
  primer
atgatcgaattcgaaatcaaataatgattttattttgactg

SEQ ID NO: 14 - DNA, 184 bp, Artificial
  DNA fragment containing attR
ctgcagtctgttacaggtcactaataccatctaagtagttgattcatagtgactgcatatgttgtgttttacagtattatgtagtctgttttttatgcaaaatctaatttaatatattgatatttatatcattttacgtttctcgttcagcttttttatactaacttgagcgtctagaaagctt

SEQ ID NO: 15 - DNA, 41 bp, Artificial
  primer
atgccactgcagtctgttacaggtcactaataccatctaag

SEQ ID NO: 16 - DNA, 46 bp, Artificial
  primer
accgttaagctttctagacgctcaagttagtataaaaaagctgaac

SEQ ID NO: 17 - DNA, 38 bp, Artificial
  primer
ttcttagacgtcaggtggcacttttcggggaaatgtgc

SEQ ID NO: 18 - DNA, 37 bp, Artificial
  primer
taacagagatctcgcgcagaaaaaaaggatctcaaga

SEQ ID NO: 19 - DNA, 46 bp, Artificial
  primer
aacagagatctaagcttagatcctttgcctggcggcagtagcgcgg

SEQ ID NO: 20 - DNA, 35 bp, Artificial
  primer
ataaactgcagcaaaaagagtttgtagaaacgcaa

SEQ ID NO: 21 - DNA, 1388 bp, Artificial
  DNA fragment containing Tc gene and ter_thrL
gaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaactagaaagcttaacacagaaaaaagcccgcacctgacagtgcgggctttttttttcgaccactgcag

SEQ ID NO: 22 - DNA, 36 bp, Artificial
  primer
agtaattctagaaagcttaacacagaaaaaagcccg

SEQ ID NO: 23 - DNA, 43 bp, Artificial
  primer
ctagtaggatccctgcagtggtcgaaaaaaaaagcccgcactg

SEQ ID NO: 24 - DNA, 1162 bp, Artificial
  DNA fragment containing Pa2 promoter
agatctccggataagtagacagcctgataagtcgcacgaaaaacaggtattgacaacatgaagtaacatgcagtaagatacaaatcgctaggtaacactagcagcgtcaaccgggcgctctagctagagccaagctagcttggccggatccgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgtcgaagcttaacacagaaaaaagcccgcacctgacagtgcgggctttttttttcgaccactgcagtctgttacaggtcactaataccatctaagtagttgattcatagtgactgcatatgttgtgttttacagtattatgtagtctgttttttatgcaaaatctaatttaatatattgatatttatatcattttacgtttctcgttcagcttttttatactaacttgagcgtctaga

SEQ ID NO: 25 - DNA, 37 bp, Artificial sequence
  primer
atcgaggtaccagatctccggataagtagacagcctg

SEQ ID NO: 26 - DNA, 32 bp, Artificial sequence
  primer
gaaggtctagagcgcccggttgacgctgctag

SEQ ID NO: 27 - DNA, 27 bp, Artificial sequence
  primer
ctaatatcgatgaagattcttgctcaa

SEQ ID NO: 28 - DNA, 34 bp, Artificial sequence
  primer
gcgttgaattccatacaacctccttagtacatgc

SEQ ID NO: 29 - DNA, 34 bp, Artificial sequence
  primer
gtactagaattcgtgtaattgcggagactttgcg

SEQ ID NO: 30 - DNA, 41 bp, Artificial
  primer
aatagcctgcagttatttgatttcaattttgtcccactccc

SEQ ID NO: 31 - DNA, 180 bp, Artificial
  DNA fragment containing PL-tac promoter
ttagatctctcacctaccaaacaatgcccccctgcaaaaaataaattcataaaaaacatacagataaccatctgcggtgataaattatctctggcggtgttgacaattaatcatcggctcgtataatgtgtggaattgtgagcgtgcttaagcttctgaacctaagaggatgctatggga

SEQ ID NO: 32 - DNA, 60 bp, Artificial
  primer
ggcatgaaatttcgattattaaagtgatggtagtcacgctcaagttagtataaaaaagct

SEQ ID NO: 33 - DNA, 40 bp, Artificial; identical to SEQ ID NO: 12
  primer

SEQ ID NO: 34 - DNA, 55 bp, Artificial
  primer
tcccatagcatcctcttaggttcagaagcttaagcacgctcacaattccacacat

SEQ ID NO: 35 - DNA, 19 bp, Artificial
  primer
agctacctctctctgattc

SEQ ID NO: 36 - DNA, 19 bp, Artificial
  primer
tgtttcccatagcatcctc

SEQ ID NO: 37 - DNA, 60 bp, Artificial
  primer
cacaacactaaacctataagttggggaaatacaatgtgaagcctgcttttttatactaag

SEQ ID NO: 38 - DNA, 60 bp, Artificial
  primer
gccgatgggcgccatttttcactgcggcaagaattacgctcaagttagtataaaaaagct

SEQ ID NO: 39 - DNA, 22 bp, Artificial
  primer
tcctggcattgattcagcctgt

SEQ ID NO: 40 - DNA, 21 bp, Artificial
  primer
ccagcagcatgagagcgatga

(19) United States
(12) Patent Application Publication
(10) Pub. No.: US 2009/0209011 A1
(43) Pub. Date: Aug. 20, 2009

(54) Method for Producing an L-Amino Acid Using a Bacterium of the Enterobacteriaceae Family With Enhanced Expression of the fucPIKUR Operon

(76) Inventors: Konstantin Vyacheslavovich Rybak, Moscow (RU); Ekaterina Aleksandrovna Slivinskaya, Moscow (RU); Marina Evgenievna Sheremet'eva, Moscow (RU); Aleksandra Yurievna Skorokhodova, Moscow (RU); Vitaly Grigorievich Paraskevov, Moscow (RU)

Correspondence Address: CERMAK & KENEALY LLP;ACS LLC, 515 EAST BRADDOCK ROAD, SUITE B, ALEXANDRIA, VA, 22314, US

(21) Appl. No.: 11/952,297

(22) Filed: Dec. 7, 2007

Related U.S. Application Data
(63) Continuation of application No. PCT/JP2006/312195, filed on Jun. 12, 2006.
(60) Provisional application No. 60/743,061, filed on Dec. 21, 2005.

(30) Foreign Application Priority Data
Jun. 17, 2005 (RU) 2005118796

(51) Int. Cl. C12P 13/04; C12P 13/24; C12P 13/22; C12P 13/20; C12P 13/14; C12P 13/06; C12P 13/08; C12P 13/12; C12N 1/21

(52) U.S. Cl. 435/107; 435/106; 435/108; 435/109; 435/110; 435/113; 435/115; 435/116; 435/252.3; 435/252.33

(57) ABSTRACT

The present invention provides a method for producing an L-amino acid using a bacterium of the Enterobacteriaceae family, particularly a bacterium belonging to the genus Escherichia or Pantoea, which has been modified to enhance expression of at least one gene of the fucPIKUR operon.

This application is a continuation under 35 U.S.C. §120 of PCT Patent Application No. PCT/JP2006/312195, filed Jun. 12, 2006, which claims priority under 35 U.S.C. §119 to Russian Patent Application No. 2005118796, filed Jun. 17, 2005, and U.S. Provisional Patent Application No. 60/743,061, filed Dec. 21, 2005. All of these documents are hereby incorporated by reference. The Sequence Listing filed electronically herewith is also hereby incorporated by reference in its entirety (File Name: US-234_Seq_List_Copy_{—}1; File Size: _KB; Date Created: Dec. 7, 2007).

### BACKGROUND OF THE INVENTION

1. Field of the Invention

The present invention relates to the microbiological industry, and specifically to a method for producing an L-amino acid using a bacterium of the Enterobacteriaceae family, which has been modified to enhance expression of genes of the fucPIKUR operon.

2. Brief Description of the Related Art

In Escherichia coli, the fucPIKUR operon is made up of five genes which encode proteins involved in utilization of L-fucose as a carbon and energy source. These genes include fucP (encoding L-fucose permease), fucI (encoding L-fucose isomerase), fucK (encoding L-fuculose kinase), fucU (encoding L-fucose-binding protein), and fucR (encoding the regulatory protein). The fucPIKUR operon is transcribed anticlockwise.

The fucP gene encodes the FucP protein, an L-fucose/proton symporter, which is also known as L-fucose permease, and is responsible for the uptake of L-fucose. FucP is the sole system of L-fucose transport in Escherichia coli (Bradley, S. A. et al., Biochem J., 1987, 248(2):495-500). The FucP protein spans the cytoplasmic membrane of E. coli 12 times, with the N- and C-termini located in the cytoplasm (Gunn, F. J. et al., Mol. Microbiol., 1995, 15(4):771-783). The FucP protein is a member of the major facilitator superfamily (MFS), which is one of the two largest families of membrane transporters and is present ubiquitously in bacteria, archaea, and eukarya (Pao, S. S. et al., Microbiol. Mol. Biol. Rev., 1998, 62(1):1-34).

The fucI gene encodes L-fucose isomerase, an enzyme of the fucose catabolism pathway, which catalyzes conversion of L-fucose to L-fuculose (Green, M. and Cohen, S. S., J. Biol. Chem., 1956, 219(2):557-568; Elsinghorst, E. A. and Mortlock, R. P., J. Bacteriol., 1994, 176(23):7223-7232).

The fucK gene encodes L-fuculose kinase (L-fuculokinase), which is an enzyme of the fucose catabolism pathway catalyzing phosphorylation of L-fuculose (Elsinghorst, E. A. and Mortlock, R. P., J. Bacteriol., 1994, 176(23):7223-7232).

The FucU protein, encoded by the fucU gene, is a cytoplasmic L-fucose binding protein which lacks any enzymatic activity on L-fucose. It was suggested that FucU may play a role in fucose transport (Kim, M. S. et al., J. Biol. Chem., 2003, 278(30):28173-28180). Utilizing NMR techniques, FucU was shown to catalyze the anomeric conversion of fucose (Ryu, K. S. et al., J. Biol. Chem., 2004, 279(24):25544-25548).

Expression of the fucPIKUR operon is controlled by the FucR protein, which is encoded by the fucR gene. FucR is a transcriptional activator that belongs to the DeoR family of transcriptional regulators (Chen, Y. M. et al., Mol. Gen. Genet., 1987, 210(2):331-337; Chen, Y. M. et al., J. Bacteriol., 1989, 171(11):6097-6105).

Currently, there have been no reports of enhancing expression of the fucPIKUR operon genes for the purpose of producing L-amino acids.

### SUMMARY OF THE INVENTION

Aspects of the present invention include enhancing the productivity of L-amino acid-producing strains and providing a method for producing an L-amino acid using these strains.

The above aspects were achieved by finding that enhancing expression of the fucPIKUR operon can increase production of L-amino acids, such as L-threonine, L-lysine, L-cysteine, L-methionine, L-leucine, L-isoleucine, L-valine, L-histidine, glycine, L-serine, L-alanine, L-asparagine, L-aspartic acid, L-glutamine, L-glutamic acid, L-proline, L-arginine, L-phenylalanine, L-tyrosine, and L-tryptophan.

The present invention provides a bacterium of the Enterobacteriaceae family having an increased ability to produce amino acids, such as L-threonine, L-lysine, L-cysteine, L-methionine, L-leucine, L-isoleucine, L-valine, L-histidine, glycine, L-serine, L-alanine, L-asparagine, L-aspartic acid, L-glutamine, L-glutamic acid, L-proline, L-arginine, L-phenylalanine, L-tyrosine, and L-tryptophan.

It is an aspect of the present invention to provide an L-amino acid-producing bacterium of the Enterobacteriaceae family, wherein the bacterium has been modified to enhance expression of at least one gene of the fucPIKUR operon.

It is a further aspect of the present invention to provide the bacterium as described above, wherein said gene expression is enhanced by modifying an expression control sequence for the fucPIKUR operon.

It is a further aspect of the present invention to provide the bacterium as described above, wherein said gene expression is enhanced by increasing the copy number for at least one gene of the fucPIKUR operon.

It is a further aspect of the present invention to provide the bacterium as described above, wherein the bacterium belongs to the genus Escherichia.

It is a further aspect of the present invention to provide the bacterium as described above, wherein the bacterium belongs to the genus Pantoea.

It is a further aspect of the present invention to provide the bacterium as described above, wherein said L-amino acid is selected from the group consisting of an aromatic L-amino acid and a non-aromatic L-amino acid.

It is a further aspect of the present invention to provide the bacterium as described above, wherein said aromatic L-amino acid is selected from the group consisting of L-phenylalanine, L-tyrosine, and L-tryptophan.

It is a further aspect of the present invention to provide the bacterium as described above, wherein said non-aromatic L-amino acid is selected from the group consisting of L-threonine, L-lysine, L-cysteine, L-methionine, L-leucine, L-isoleucine, L-valine, L-histidine, glycine, L-serine, L-alanine, L-asparagine, L-aspartic acid, L-glutamine, L-glutamic acid, L-proline, and L-arginine.

It is a further aspect of the present invention to provide a method for producing an L-amino acid comprising:
cultivating the bacterium as described above in a medium to produce and excrete said L-amino acid into the medium, and
collecting said L-amino acid from the medium.

It is a further aspect of the present invention to provide the method as described above, wherein said L-amino acid is selected from the group consisting of an aromatic L-amino acid and a non-aromatic L-amino acid.

It is a further aspect of the present invention to provide the method as described above, wherein said aromatic L-amino acid is selected from the group consisting of L-phenylalanine, L-tyrosine, and L-tryptophan.

It is a further aspect of the present invention to provide the method as described above, wherein said non-aromatic L-amino acid is selected from the group consisting of L-threonine, L-lysine, L-cysteine, L-methionine, L-leucine, L-isoleucine, L-valine, L-histidine, glycine, L-serine, L-alanine, L-asparagine, L-aspartic acid, L-glutamine, L-glutamic acid, L-proline, and L-arginine.

The present invention is described in detail below.

### BRIEF DESCRIPTION OF THE DRAWINGS

FIG. 1 shows the construction of the pMW118-attL-Cm-attR plasmid, which is used as a template for PCR.

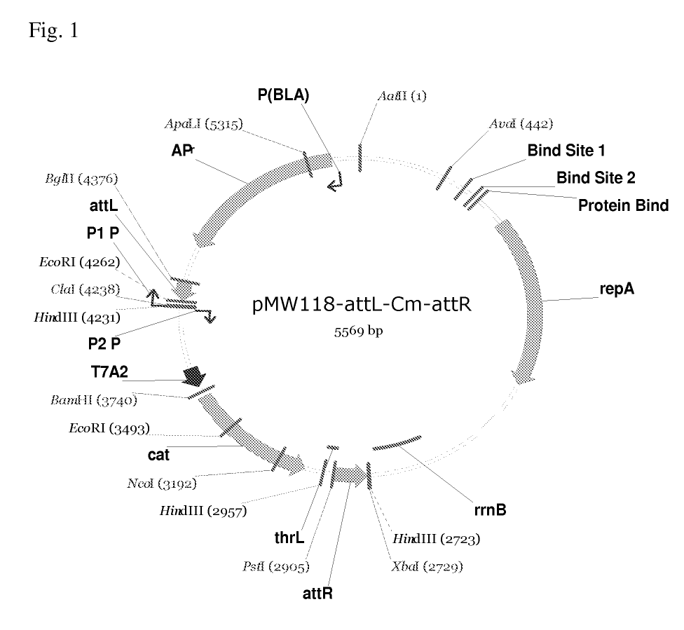

FIG. 2 shows the relative positions of primers P17 and P18 on plasmid pMW118-attL-Cm-attR, which are used for PCR amplification of the cat gene.

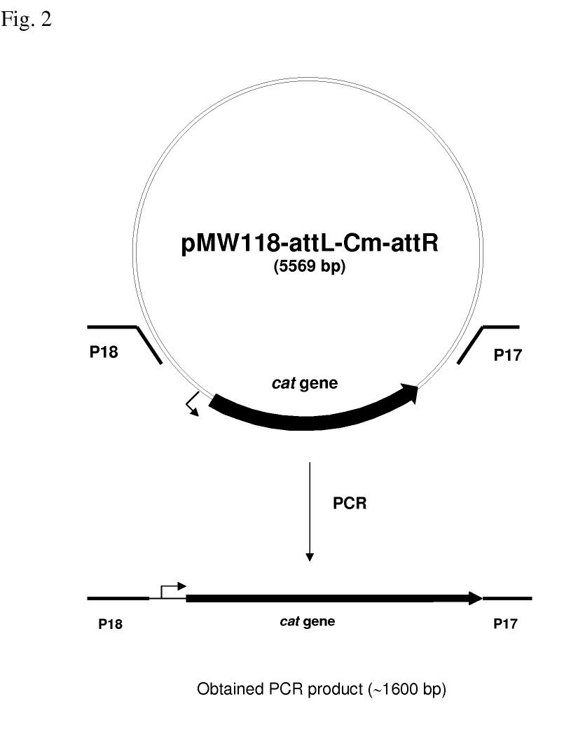

FIG. 3 shows the construction of the chromosomal DNA fragment containing the hybrid P_{L-tac} promoter.

FIG. 4 shows the effect of enhanced expression of the fucPIKUR operon on growth of E. coli with a disrupted PTS transport system.

### DESCRIPTION OF THE PREFERRED EMBODIMENTS

### 1. Bacterium of the Present Invention

The bacterium of the present invention is an L-amino acid-producing bacterium of the Enterobacteriaceae family, wherein the bacterium has been modified to enhance expression of at least one gene involved in utilization of fucose, especially expression of the fucPIKUR operon.

In the present invention, “L-amino acid-producing bacterium” means a bacterium which has an ability to produce and excrete an L-amino acid into a medium, when the bacterium is cultured in the medium.

The term “L-amino acid-producing bacterium” as used herein also means a bacterium which is able to produce and cause accumulation of an L-amino acid in a culture medium in an amount larger than a wild-type or parental strain of the bacterium, for example, E. coli, such as E. coli K-12, and preferably means that the bacterium is able to produce of the target L-amino acid in a medium an amount not less than 0.5 g/L, more preferably not less than 1.0 g/L. The term “L-amino acid” includes L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, and L-valine.

The term “aromatic L-amino acid” includes L-phenylalanine, L-tyrosine, and L-tryptophan. The term “non-aromatic L-amino acid” includes L-threonine, L-lysine, L-cysteine, L-methionine, L-leucine, L-isoleucine, L-valine, L-histidine, glycine, L-serine, L-alanine, L-asparagine, L-aspartic acid, L-glutamine, L-glutamic acid, L-proline, and L-arginine. L-threonine, L-lysine, L-cysteine, L-leucine, L-histidine, L-glutamic acid, L-phenylalanine, L-tryptophan, L-proline and L-arginine are particularly preferred.

The Enterobacteriaceae family includes bacteria belonging to the genera Escherichia, Enterobacter, Erwinia, Klebsiella, Pantoea, Photorhabdus, Providencia, Salmonella, Serratia, Shigella, Morganella Yersinia, etc. Specifically, those classified into the Enterobacteriaceae according to the taxonomy used by the NCBI (National Center for Biotechnology Information) database (http://www.ncbi.nlm.nih.gov/Taxonomy/Browser/wwwtax.cgi?id=91347) can be used. A bacterium belonging to the genus Escherichia or Pantoea is preferred.

The phrase “a bacterium belonging to the genus Escherichia” means that the bacterium is classified into the genus Escherichia according to the classification known to a person skilled in the art of microbiology. Examples of a bacterium belonging to the genus Escherichia include, but are not limited to, Escherichia coli (E. coli).

The bacterium belonging to the genus Escherichia that can be used in the present invention is not particularly limited; however, e.g., bacteria described by Neidhardt, F. C. et al. (Escherichia coli and Salmonella typhimurium, American Society for Microbiology, Washington D.C., 1208, Table 1) are encompassed by the present invention.

The phrase “a bacterium belonging to the genus Pantoea” means that the bacterium is classified into the genus Pantoea according to the classification known to a person skilled in the art of microbiology. Some species of Enterobacter agglomerans have been recently re-classified into Pantoea agglomerans, Pantoea ananatis, Pantoea stewartii or the like, based on the nucleotide sequence analysis of 16S rRNA, etc (Int. J. Syst. Bacteriol., 43, 162-173 (1993)).

The phrase “bacterium has been modified to enhance expression of the fucPIKUR operon” means that the bacterium has been modified in such a way that the modified bacterium contains increased amounts of the proteins FucP, FucI, FucK, FucU, and FucR, as compared with an unmodified bacterium.

The phrase “the enhanced expression of the fucPIKUR operon” means that the expression levels of the genes of the fucPIKUR operon are higher than that of a non-modified strain, for example, a wild-type strain. Examples of modifications include increasing the copy number of the expressed gene(s) per cell, and/or increasing the expression level of the gene(s) by modification of an adjacent region of the gene, including sequences controlling gene expression, such as a promoter, enhancer, attenuator, ribosome-binding site, etc.

The fucP gene encodes the FucP protein, an L-fucose/proton symporter (synonym—B2801). The fucP gene of E. coli (nucleotide positions: 2,932,257 to 2,933,573; GenBank accession no. NC_{—}000913.2; gi:49175990; SEQ ID NO: 1) is located between the fucA and fucI genes on the chromosome of E. coli K-12. The nucleotide sequence of the fucP gene and the amino acid sequence of the FucP protein encoded by the fucP gene are shown in SEQ ID NO: 1 and SEQ ID NO: 2, respectively.

The fucI gene encodes the FucK protein, which is an L-fucose isomerase (synonym—B2802). The fucI gene of E. coli (nucleotide positions: 2,933,606 to 2,935,381; GenBank accession no. NC_{—}000913.2; gi:49175990; SEQ ID NO: 3) is located between the fucP and fucK genes on the chromosome of E. coli K-12. The nucleotide sequence of the fucI gene and the amino acid sequence of the FucI protein encoded by the fucI gene are shown in SEQ ID NO: 3 and SEQ ID NO: 4, respectively.

The fucK gene encodes the FucK protein, which is an L-fuculokinase (synonyms—B2803, ATP:L-fuculose 1-phosphotransferase). The fucK gene of E. coli (nucleotide positions: 2,935,460 to 2,936,908; GenBank accession no. NC_{—}000913.2; gi:49175990; SEQ ID NO: 5) is located between the fucI and fucU genes on the chromosome of E. coli K-12. The nucleotide sequence of the fucI gene and the amino acid sequence of the FucK protein encoded by the fucK gene are shown in SEQ ID NO: 5 and SEQ ID NO: 6, respectively.

The fucU gene encodes the FucU protein, which is a cytoplasmic L-fucose-binding protein (synonym—B2804). The fucU gene of E. coli (nucleotide positions: 2,936,910 to 2,937,332; GenBank accession no. NC_{—}000913.2; gi:49175990; SEQ ID NO: 7) is located between the fucK and fucR genes on the chromosome of E. coli K-12. The nucleotide sequence of the fucU gene and the amino acid sequence of the FucU protein encoded by the fucU gene are shown in SEQ ID NO: 7 and SEQ ID NO: 8, respectively.

The fucR gene encodes the FucR protein, which is a transcriptional activator (synonyms—B2805, positive regulator of the fuc operon). The fucR gene of E. coli (nucleotide positions: 2,937,390 to 2,938,121; GenBank accession no. NC_{—}000913.2; gi:49175990; SEQ ID NO: 9) is located between the fucU and ygdE genes on the chromosome of E. coli K-12. The nucleotide sequence of the fucR gene and the amino acid sequence of the FucR protein encoded by the fucR gene are shown in SEQ ID NO: 9 and SEQ ID NO: 10, respectively.

The genes of the fucPIKUR operon can be obtained by PCR (polymerase chain reaction; refer to White, T. J. et al., Trends Genet., 5, 185 (1989)) utilizing primers prepared based on the known nucleotide sequence of the gene.

Since there may be some differences in DNA sequences between the genera or strains of the Enterobacteriaceae family, the above-described genes of the fucPIKUR operon to be overexpressed are not limited to the nucleotide sequences shown in SEQ ID NOS: 1, 3, 5, 7 and 9, but may also include nucleotide sequences homologous to SEQ ID NOS: 1, 3, 5, 7 and 9 encoding variant proteins of the FucP, FucI, FucK, FucU and FucR proteins, respectively. The phrase “variant protein” as used in the present invention means a protein which has changes in the sequence, whether they are deletions, insertions, additions, or substitutions of one or several amino acids, but still maintains the activity of the product as the FucP, FucI, FucK, FucU or FucR protein. The number of changes in the variant protein depends on the position in the three dimensional structure of the protein or the type of amino acid residues. It may be 1 to 30, preferably 1 to 15, and more preferably 1 to 5 in SEQ ID NO: 2, 4, 6, 8 or 10. These changes in the variants can occur in regions of the protein which are not critical for the function of the protein. This is because some amino acids have high homology to one another so the three dimensional structure or activity is not affected by such a change. These changes in the variant protein can occur in regions of the protein which are not critical for the function of the protein. Therefore, the protein variants encoded by the above-described genes of the fucPIKUR operon may have a similarity (homology) of not less than 80%, preferably not less than 90%, and most preferably not less than 95%, with respect to the entire amino acid sequences shown in SEQ ID NOS. 2, 4, 6, 8 or 10, as long as the ability of the proteins to utilize L-fucose is maintained. Homology between two amino acid sequences can be determined using the well-known methods, for example, the computer program BLAST 2.0, which calculates three parameters: score, identity and similarity.

The substitution, deletion, insertion, or addition of one or several amino acid residues should be conservative mutation(s) so that the activity is maintained. The representative conservative mutation is a conservative substitution. Examples of conservative substitutions include substitution of Ser or Thr for Ala, substitution of Gln, His or Lys for Arg, substitution of Glu, Gln, Lys, His or Asp for Asn, substitution of Asn, Glu or Gln for Asp, substitution of Ser or Ala for Cys, substitution of Asn, Glu, Lys, His, Asp or Arg for Gln, substitution of Asn, Gln, Lys or Asp for Glu, substitution of Pro for Gly, substitution of Asn, Lys, Gln, Arg or Tyr for His, substitution of Leu, Met, Val or Phe for Ile, substitution of Ile, Met, Val or Phe for Leu, substitution of Asn, Glu, Gln, His or Arg for Lys, substitution of Ile, Leu, Val or Phe for Met, substitution of Trp, Tyr, Met, Ile or Leu for Phe, substitution of Thr or Ala for Ser, substitution of Ser or Ala for Thr, substitution of Phe or Tyr for Trp, substitution of His, Phe or Trp for Tyr, and substitution of Met, Ile or Leu for Val.

Moreover, each of the above-described genes of the fucPIKUR operon may be a variant which hybridizes under stringent conditions with the nucleotide sequence shown in SEQ ID NOS: 1, 3, 5, 7 or 9, or with a probe which can be prepared from the nucleotide sequence, provided that it encodes a functional protein. “Stringent conditions” include those under which a specific hybrid, for example, a hybrid having homology of not less than 60%, preferably not less than 70%, more preferably not less than 80%, still more preferably not less than 90%, and most preferably not less than 95%, is formed and a non-specific hybrid, for example, a hybrid having homology lower than the above, is not formed. For example, stringent conditions are exemplified by washing at 60° C. one time or more, preferably two or three times, at a salt concentration of 1×SSC and 0.1% SDS, preferably 0.1×SSC and 0.1% SDS at 60° C. Duration of washing depends on the type of membrane used for blotting and, as a rule, may be what is recommended by the manufacturer. For example, the recommended duration of washing for the Hybond™ N⁺ nylon membrane (Amersham) under stringent conditions is 15 minutes. Preferably, washing may be performed 2 to 3 times. The length of the probe may be suitably selected, depending on the hybridization conditions, and usually varies from 100 bp to 1 kbp.

Methods of enhancing gene expression include increasing the gene copy number. Introduction of a recombinant plasmid comprising the gene and a vector that is able to function in a bacterium of the Enterobacteriaceae family increases the copy number of the gene. Low copy vectors and high copy vectors may be used. However, low copy vectors are preferably used. Examples of low-copy vectors include but are not limited to pSC101, pMW118, pMW119, and the like. The term “low copy vector” indicates vectors which are present in an amount of up to 5 copies per cell.

Enhancing gene expression may also be achieved by introducing multiple copies of the gene into the bacterial chromosome by, for example, homologous recombination, Mu integration, or the like. For example, one act of Mu integration allows for introduction of up to 3 copies of the gene into the bacterial chromosome.

Increasing the copy number of genes of the fucPIKUR operon can also be achieved by introducing multiple copies of the genes into the chromosomal DNA of the bacterium. In order to introduce multiple copies of the gene into the bacterial chromosome, homologous recombination is carried out using a target sequence present in multiple copies on the chromosomal DNA. Sequences having multiple copies in the chromosomal DNA include, but are not limited to, repetitive DNA, or inverted repeats present at the end of a transposable element. Also, as disclosed in U.S. Pat. No. 5,595,889, it is possible to incorporate genes of the fucPIKUR operon into a transposon, and allow it to be transferred to introduce multiple copies of the genes into the chromosomal DNA.

Enhancing gene expression may also be achieved by placing genes of the fucPIKUR operon under the control of a strong promoter which is preferably stronger than the native promoter of the operon. For example, the P_{tac} promoter, the lac promoter, the trp promoter, the trc promoter, the P_{R}, or the P_{L} promoters of lambda phage are all known to be strong promoters. The use of a strong promoter can be combined with multiplication of gene copies.

Alternatively, the effect of a promoter can be enhanced by, for example, introducing a mutation into the promoter to increase the transcription level of a gene located downstream of the promoter. Furthermore, it is known that substitution of several nucleotides in the spacer region between the ribosome binding site (RBS) and the start codon, especially the sequences immediately upstream of the start codon, profoundly affect the mRNA translatability. For example, a 20-fold range in the expression levels was found, depending on the nature of the three nucleotides preceding the start codon (Gold et al., Annu. Rev. Microbiol., 35, 365-403, 1981; Hui et al., EMBO J., 3, 623-629, 1984). Previously, it was shown that the rhtA23 mutation, which increases the resistance to threonine, homoserine and some other substances transported out of cells, is an A-for-G substitution at the −1 position relative to the ATG start codon (ABSTRACTS of 17th International Congress of Biochemistry and Molecular Biology in conjugation with 1997 Annual Meeting of the American Society for Biochemistry and Molecular Biology, San Francisco, Calif. Aug. 24-29, 1997, abstract No. 457). Therefore, it may be suggested that the rhtA23 mutation enhances translation of the rhtA gene transcript and, as a consequence, increases the resistance to the above-mentioned substances.

Moreover, it is also possible to introduce a nucleotide substitution into the expression control sequence, such as a promoter region of the fucPIKUR operon, on the bacterial chromosome, which results in stronger promoter function. The alteration of the expression control sequence can be performed, for example, in the same manner as the gene substitution using a temperature-sensitive plasmid, as disclosed in WO 00/18935 and JP 1-215280 A.

The level of gene expression can be determined by measuring the amount of mRNA transcribed from the gene using various well-known methods, including Northern blotting, quantitative RT-PCR, and the like. The amount or molecular weight of the protein encoded by the gene can be measured by well-known methods, including SDS-PAGE followed by immunoblotting assay (Western blotting analysis) and the like.

Methods for preparation of plasmid DNA, digestion and ligation of DNA, transformation, selection of an oligonucleotide as a primer, and the like may be ordinary methods well-known to one skilled in the art. These methods are described, for instance, in Sambrook, J., Fritsch, E. F., and Maniatis, T., “Molecular Cloning: A Laboratory Manual, Second Edition”, Cold Spring Harbor Laboratory Press (1989).

### L-Amino Acid-Producing Bacteria

As a bacterium of the present invention which is modified to enhance expression of the fucPIKUR operon, bacteria which are able to produce either an aromatic or a non-aromatic L-amino acid may be used.

The bacterium of the present invention can be obtained by enhancing expression of the fucPIKUR operon in a bacterium, which inherently has the ability to produce an L-amino acid. Alternatively, the bacterium of present invention can be obtained by imparting the ability to produce an L-amino acid to a bacterium already having the expression of the fucPIKUR operon enhanced.

### L-Threonine-Producing Bacteria

Examples of parent strains for deriving the L-threonine-producing bacteria of the present invention include, but are not limited to, strains belonging to the genus Escherichia, such as E. coli TDH-6/pVIC40 (VKPM B-3996) (U.S. Pat. No. 5,175,107, U.S. Pat. No. 5,705,371), E. coli 472T23/pYN7 (ATCC 98081) (U.S. Pat. No. 5,631,157), E. coli NRRL-21593 (U.S. Pat. No. 5,939,307), E. coli FERM BP-3756 (U.S. Pat. No. 5,474,918), E. coli FERM BP-3519 and FERM BP-3520 (U.S. Pat. No. 5,376,538), E. coli MG442 (Gusyatiner et al., Genetika (in Russian), 14, 947-956 (1978)), E. coli VL643 and VL2055 (EP 1149911 A), and the like.

The strain TDH-6 is deficient in the thrC gene, as well as being sucrose-assimilative, and the ilvA gene has a leaky mutation. This strain also has a mutation in the rhtA gene, which imparts resistance to high concentrations of threonine or homoserine. The strain B-3996 contains the plasmid pVIC40 which was obtained by inserting a thrA*BC operon which includes a mutant thrA gene into a RSF1010-derived vector. This mutant thrA gene encodes aspartokinase homoserine dehydrogenase I which is substantially desensitized to feedback inhibition by threonine. The strain B-3996 was deposited on Nov. 19, 1987 in the All-Union Scientific Center of Antibiotics (Nagatinskaya Street 3-A, 117105 Moscow, Russian Federation) under the accession number RIA 1867. The strain was also deposited in the Russian National Collection of Industrial Microorganisms (VKPM) (Russia, 117545 Moscow, 1 Dorozhny proezd. 1) on Apr. 7, 1987 under the accession number VKPM B-3996.

E. coli VKPM B-5318 (EP 0593792B) may also be used as a parent strain for deriving L-threonine-producing bacteria of the present invention. The strain B-5318 is prototrophic with regard to isoleucine, and a temperature-sensitive lambda-phage C1 repressor and PR promoter replaces the regulatory region of the threonine operon in plasmid pVIC40. The strain VKPM B-5318 was deposited in the Russian National Collection of Industrial Microorganisms (VKPM) on May 3, 1990 under accession number of VKPM B-5318.

Preferably, the bacterium of the present invention is additionally modified to enhance expression of one or more of the following genes:
the mutant thrA gene which codes for aspartokinase homoserine dehydrogenase I resistant to feed back inhibition by threonine;
the thrB gene which codes for homoserine kinase;
the thrC gene which codes for threonine synthase;
the rhtA gene which codes for a putative transmembrane protein;
the asd gene which codes for aspartate-β-semialdehyde dehydrogenase; and the aspC gene which codes for aspartate aminotransferase (aspartate transaminase);

The thrA gene which encodes aspartokinase homoserine dehydrogenase I of Escherichia coli has been elucidated (nucleotide positions 337 to 2799, GenBank accession NC_{—}000913.2, gi: 49175990). The thrA gene is located between the thrL and thrB genes on the chromosome of E. coli K-12. The thrB gene which encodes homoserine kinase of Escherichia coli has been elucidated (nucleotide positions 2801 to 3733, GenBank accession NC_{—}000913.2, gi: 49175990). The thrB gene is located between the thrA and thrC genes on the chromosome of E. coli K-12. The thrC gene which encodes threonine synthase of Escherichia coli has been elucidated (nucleotide positions 3734 to 5020, GenBank accession NC_{—}000913.2, gi: 49175990). The thrC gene is located between the thrB gene and the yaaX open reading frame on the chromosome of E. coli K-12. All three genes functions as a single threonine operon. To enhance expression of the threonine operon, the attenuator region which affects the transcription is desirably removed from the operon (WO2005/049808, WO2003/097839).

A mutant thrA gene which codes for aspartokinase homoserine dehydrogenase I resistant to feedback inhibition by threonine, as well as, the thrB and thrC genes can be obtained as one operon from the well-known plasmid pVIC40, which is present in the threonine producing E. coli strain VKPM B-3996. Plasmid pVIC40 is described in detail in U.S. Pat. No. 5,705,371.

The rhtA gene exists at 18 min on the E. coli chromosome close to the glnHPQ operon, which encodes components of the glutamine transport system. The rhtA gene is identical to ORF1 (ybiF gene, nucleotide positions 764 to 1651, GenBank accession number AAA218541, gi:440181) and located between the pexB and ompX genes. The unit expressing a protein encoded by the ORF1 has been designated the rhtA gene (rht: resistance to homoserine and threonine). Also, it was revealed that the rhtA23 mutation is an A-for-G substitution at position −1 with respect to the ATG start codon (ABSTRACTS of the 17th International Congress of Biochemistry and Molecular Biology in conjugation with Annual Meeting of the American Society for Biochemistry and Molecular Biology, San Francisco, Calif. Aug. 24-29, 1997, abstract No. 457, EP 1013765 A).

The asd gene of E. coli has already been elucidated (nucleotide positions 3572511 to 3571408, GenBank accession NC_{—}000913.1, gi:16131307), and can be obtained by PCR (polymerase chain reaction; refer to White, T. J. et al., Trends Genet., 5, 185 (1989)) utilizing primers prepared based on the nucleotide sequence of the gene. The asd genes of other microorganisms can be obtained in a similar manner.

Also, the aspC gene of E. coli has already been elucidated (nucleotide positions 983742 to 984932, GenBank accession NC_{—}000913.1, gi:16128895), and can be obtained by PCR. The aspC genes of other microorganisms can be obtained in a similar manner.

### L-Lysine-Producing Bacteria

Examples of L-lysine-producing bacteria belonging to the genus Escherichia include mutants having resistance to an L-lysine analogue. The L-lysine analogue inhibits growth of bacteria belonging to the genus Escherichia, but this inhibition is fully or partially desensitized when L-lysine is present in the medium. Examples of the L-lysine analogue include, but are not limited to, oxalysine, lysine hydroxamate, S-(2-aminoethyl)-L-cysteine (AEC), γ-methyllysine, α-chlorocaprolactam, and so forth. Mutants having resistance to these lysine analogues can be obtained by subjecting bacteria belonging to the genus Escherichia to a conventional artificial mutagenesis treatment. Specific examples of bacterial strains useful for producing L-lysine include Escherichia coli AJ11442 (FERM BP-1543, NRRL B-12185; see U.S. Pat. No. 4,346,170) and Escherichia coli VL611. In these microorganisms, feedback inhibition of aspartokinase by L-lysine is desensitized.

The strain WC196 may be used as an L-lysine producing bacterium of Escherichia coli. This bacterial strain was bred by conferring AEC resistance to the strain W3110, which was derived from Escherichia coli K-12. The resulting strain was designated Escherichia coli AJ13069 strain and was deposited at the National Institute of Bioscience and Human-Technology, Agency of Industrial Science and Technology (currently National Institute of Advanced Industrial Science and Technology, International Patent Organism Depositary, Tsukuba Central 6, 1-1, Higashi 1-Chome, Tsukuba-shi, Ibaraki-ken, 305-8566, Japan) on Dec. 6, 1994 and received an accession number of FERM P-14690. Then, it was converted to an international deposit under the provisions of the Budapest Treaty on Sep. 29, 1995, and received an accession number of FERM BP-5252 (U.S. Pat. No. 5,827,698).

Examples of parent strains for deriving L-lysine-producing bacteria of the present invention also include strains in which expression of one or more genes encoding an L-lysine biosynthetic enzyme are enhanced. Examples of such genes include, but are not limited to, genes encoding dihydrodipicolinate synthase (dapA), aspartokinase (lysC), dihydrodipicolinate reductase (dapB), diaminopimelate decarboxylase (lysA), diaminopimelate dehydrogenase (ddh) (U.S. Pat. No. 6,040,160), phosphoenolpyrvate carboxylase (ppc), aspartate semialdehyde dehydrogenease (asd), and aspartase (aspA) (EP 1253195 A). In addition, the parent strains may have an increased level of expression of the gene involved in energy efficiency (cyo) (EP 1170376 A), the gene encoding nicotinamide nucleotide transhydrogenase (pntAB) (U.S. Pat. No. 5,830,716), the ybjE gene (WO2005/073390), or combinations thereof.

Examples of parent strains for deriving L-lysine-producing bacteria of the present invention also include strains with decreased or no activity of an enzyme that catalyzes a reaction for generating a compound other than L-lysine by branching off from the biosynthetic pathway of L-lysine. Examples of the enzymes that catalyze a reaction for generating a compound other than L-lysine by branching off from the biosynthetic pathway of L-lysine include homoserine dehydrogenase, lysine decarboxylase (U.S. Pat. No. 5,827,698), and the malic enzyme (WO2005/010175).

### L-Cysteine-Producing Bacteria

Examples of parent strains for deriving L-cysteine-producing bacteria of the present invention include, but are not limited to, strains belonging to the genus Escherichia, such as E. coli JM15 which is transformed with different cysE alleles coding for feedback-resistant serine acetyltransferases (U.S. Pat. No. 6,218,168, Russian patent application 2003121601); E. coli W3110 with over-expressed genes which encode proteins suitable for secreting substances toxic for cells (U.S. Pat. No. 5,972,663); E. coli strains with reduced cysteine desulfohydrase activity (JP11155571A2); E. coli W3110 with increased activity of a positive transcriptional regulator for cysteine regulon encoded by the cysB gene (WO0127307A1), and the like.

### L-Leucine-Producing Bacteria

Examples of parent strains for deriving L-leucine-producing bacteria of the present invention include, but are not limited to, strains belonging to the genus Escherichia, such as E. coli strains resistant to leucine (for example, the strain 57 (VKPM B-7386, U.S. Pat. No. 6,124,121)) or leucine analogs including β-2-thienylalanine, 3-hydroxyleucine, 4-azaleucine, 5,5,5-trifluoroleucine (JP 62-34397 B and JP 8-70879 A); E. coli strains obtained by the gene engineering method described in WO96/06926; E. coli H-9068 (JP 8-70879 A), and the like.

The bacterium of the present invention may be improved by enhancing the expression of one or more genes involved in L-leucine biosynthesis. Examples include genes of the leuABCD operon, which are preferably represented by a mutant leuA gene coding for isopropylmalate synthase which is not subject to feedback inhibition by L-leucine (U.S. Pat. No. 6,403,342). In addition, the bacterium of the present invention may be improved by enhancing the expression of one or more genes coding for proteins which excrete L-amino acid from the bacterial cell. Examples of such genes include the b2682 and b2683 genes (ygaZH genes) (EP 1239041 A2).

### L-Histidine-Producing Bacteria

Examples of parent strains for deriving L-histidine-producing bacteria of the present invention include, but are not limited to, strains belonging to the genus Escherichia, such as E. coli strain 24 (VKPM B-5945, RU2003677); E. coli strain 80 (VKPM B-7270, RU2119536); E. coli NRRL B-12116-B12121 (U.S. Pat. No. 4,388,405); E. coli H-9342 (FERM BP-6675) and H-9343 (FERM BP-6676) (U.S. Pat. No. 6,344,347); E. coli H-9341 (FERM BP-6674) (EP1085087); E. coli A180/pFM201 (U.S. Pat. No. 6,258,554) and the like.

Examples of parent strains for deriving L-histidine-producing bacteria of the present invention also include strains in which expression of one or more genes encoding an L-histidine biosynthetic enzyme are enhanced. Examples of such genes include genes encoding ATP phosphoribosyltransferase (hisG), phosphoribosyl AMP cyclohydrolase (hisI), phosphoribosyl-ATP pyrophosphohydrolase (hisIE), phosphoribosylformimino-5-aminoimidazole carboxamide ribotide isomerase (hisA), amidotransferase (hisH), histidinol phosphate aminotransferase (hisC), histidinol phosphatase (hisB), histidinol dehydrogenase (hisD), and so forth.

It is known that the L-histidine biosynthetic enzymes encoded by hisG and hisBHAFI are inhibited by L-histidine, and therefore an L-histidine-producing ability can also be efficiently enhanced by introducing a mutation conferring resistance to the feedback inhibition into ATP phosphoribosyltransferase (Russian Patent Nos. 2003677 and 2119536).

Specific examples of strains having an L-histidine-producing ability include E. coli FERM-P 5038 and 5048 which have been transformed with a vector carrying a DNA encoding an L-histidine-biosynthetic enzyme (JP 56-005099 A), E. coli strains transformed with rht, a gene for an amino acid-export (EP1016710A), E. coli 80 strain imparted with sulfaguanidine, DL-1,2,4-triazole-3-alanine, and streptomycin-resistance (VKPM B-7270, Russian Patent No. 2119536), and so forth.

### L-Glutamic Acid-Producing Bacteria

Examples of parent strains for deriving L-glutamic acid-producing bacteria of the present invention include, but are not limited to, strains belonging to the genus Escherichia, such as E. coli VL334thrC⁺ (EP 1172433). E. coli VL334 (VKPM B-1641) is an L-isoleucine and L-threonine auxotrophic strain having mutations in thrC and ilvA genes (U.S. Pat. No. 4,278,765). A wild-type allele of the thrC gene was transferred by the method of general transduction using a bacteriophage P1 grown on the wild-type E. coli strain K12 (VKPM B-7) cells. As a result, an L-isoleucine auxotrophic strain VL334thrC⁺ (VKPM B-8961), which is able to produce L-glutamic acid, was obtained.

Examples of parent strains for deriving the L-glutamic acid-producing bacteria of the present invention include, but are not limited to, strains in which expression of one or more genes encoding an L-glutamic acid biosynthetic enzyme are enhanced. Examples of such genes include genes encoding glutamate dehydrogenase (gdh), glutamine synthetase (glnA), glutamate synthetase (gltAB), isocitrate dehydrogenase (icdA), aconitate hydratase (acnA, acnB), citrate synthase (gltA), phosphoenolpyruvate carboxylase (ppc), pyruvate dehydrogenase (aceEF, lpdA), pyruvate kinase (pykA, pykF), phosphoenolpyruvate synthase (ppsA), enolase (eno), phosphoglyceromutase (pgmA, pgmI), phosphoglycerate kinase (pgk), glyceraldehyde-3-phophate dehydrogenase (gapA), triose phosphate isomerase (tpiA), fructose bisphosphate aldolase (fbp), phosphofructokinase (pfkA, pfkB), and glucose phosphate isomerase (pgi).

Examples of strains modified so that expression of the citrate synthetase gene, the phosphoenolpyruvate carboxylase gene, and/or the glutamate dehydrogenase gene is/are enhanced include those disclosed in EP1078989A, EP955368A, and EP952221A.

Examples of parent strains for deriving the L-glutamic acid-producing bacteria of the present invention also include strains with decreased or no activity of an enzyme that catalyzes synthesis of a compound other than L-glutamic acid by branching off from an L-glutamic acid biosynthesis pathway. Examples of such genes include genes encoding isocitrate lyase (aceA), α-ketoglutarate dehydrogenase (sucA), phosphotransacetylase (pta), acetate kinase (ack), acetohydroxy acid synthase (ilvG), acetolactate synthase (ilvI), formate acetyltransferase (pfl), lactate dehydrogenase (ldh), and glutamate decarboxylase (gadAB). Bacteria belonging to the genus Escherichia deficient in the α-ketoglutarate dehydrogenase activity or having a reduced α-ketoglutarate dehydrogenase activity and methods for obtaining them are described in U.S. Pat. Nos. 5,378,616 and 5,573,945. Specifically, these strains include the following:

E. coli W3110sucA::Kmr

E. coli AJ12624 (FERM BP-3853)

E. coli AJ12628 (FERM BP-3854)

E. coli AJ12949 (FERM BP-4881)

E. coli W310sucA::Kmr is a strain obtained by disrupting the α-ketoglutarate dehydrogenase gene (hereinafter referred to as “sucA gene”) of E. coli W3110. This strain is completely deficient in the α-ketoglutarate dehydrogenase.

Other examples of L-glutamic acid-producing bacterium include those which belong to the genus Escherichia and have resistance to an aspartic acid antimetabolite. These strains can also be deficient in α-ketoglutarate dehydrogenase activity and include, for example, E. coli AJ13199 (FERM BP-5807) (U.S. Pat. No. 5,908,768), FFRM P-12379, which additionally has a low L-glutamic acid decomposing ability (U.S. Pat. No. 5,393,671); AJ13138 (FERM BP-5565) (U.S. Pat. No. 6,110,714), and the like.

Examples of L-glutamic acid-producing bacteria, include mutant strains belonging to the genus Pantoea which are deficient in α-ketoglutarate dehydrogenase activity or have a decreased α-ketoglutarate dehydrogenase activity, and can be obtained as described above. Such strains include Pantoea ananatis AJ13356. (U.S. Pat. No. 6,331,419). Pantoea ananatis AJ13356 was deposited at the National Institute of Bioscience and Human-Technology, Agency of Industrial Science and Technology, Ministry of International Trade and Industry (currently, National Institute of Advanced Industrial Science and Technology, International Patent Organism Depositary, Central 6, 1-1, Higashi 1-Chome, Tsukuba-shi, Ibaraki-ken, 305-8566, Japan) on Feb. 19, 1998 under an accession number of FERM P-16645. It was then converted to an international deposit under the provisions of Budapest Treaty on Jan. 11, 1999 and received an accession number of FERM BP-6615. Pantoea ananatis AJ13356 is deficient in α-ketoglutarate dehydrogenase activity as a result of disruption of the αKGDH-E1 subunit gene (sucA). The above strain was identified as Enterobacter agglomerans when it was isolated and deposited as the Enterobacter agglomerans AJ13356. However, it was recently re-classified as Pantoea ananatis on the basis of nucleotide sequencing of 16S rRNA and so forth. Although AJ13356 was deposited at the aforementioned depository as Enterobacter agglomerans, for the purposes of this specification, they are described as Pantoea ananatis.

### L-Phenylalanine-Producing Bacteria

Examples of parent strains for deriving L-phenylalanine-producing bacteria of the present invention include, but are not limited to, strains belonging to the genus Escherichia, such as E. coli AJ12739 (tyrA::Tn10, tyrR) (VKPM B-8197); E. coli HW1089 (ATCC 55371) harboring the mutant pheA34 gene (U.S. Pat. No. 5,354,672); E. coli MWEC101-b (KR8903681); E. coli NRRL B-12141, NRRL B-12145, NRRL B-12146 and NRRL B-12147 (U.S. Pat. No. 4,407,952). Also, as a parent strain, E. coli K-12 [W3110 (tyrA)/pPHAB (FERM BP-3566), E. coli K-12 [W3110 (tyrA)/pPHAD] (FERM BP-12659), E. coli K-12 [W3110 (tyrA)/pPHATerm] (FERM BP-12662) and E. coli K-12 [W3110 (tyrA)/pBR-aroG4, pACMAB] named as AJ 12604 (FERM BP-3579) may be used (EP 488-424 B1). Furthermore, L-phenylalanine producing bacteria belonging to the genus Escherichia with an enhanced activity of the protein encoded by the yedA gene or the yddG gene may also be used (U.S. patent applications 2003/0148473 A1 and 2003/0157667 A1).

L-Tryptophan-Producing Bacteria

Examples of parent strains for deriving the L-tryptophan-producing bacteria of the present invention include, but are not limited to, strains belonging to the genus Escherichia, such as E. coli JP4735/pMU3028 (DSM10122) and JP6015/pMU91 (DSM10123) which is deficient in the tryptophanyl-tRNA synthetase encoded by mutant trpS gene (U.S. Pat. No. 5,756,345); E. coli SV164 (pGH5) having a serA allele encoding phosphoglycerate dehydrogenase not subject to feedback inhibition by serine and a trpE allele encoding anthranilate synthase not subject to feedback inhibition by tryptophan (U.S. Pat. No. 6,180,373); E. coli AGX17 (pGX44) (NRRL B-12263) and AGX6(pGX50)aroP (NRRL B-12264) which is deficient in the enzyme tryptophanase (U.S. Pat. No. 4,371,614); E. coli AGX17/pGX50,pACKG4-pps in which a phosphoenolpyruvate-producing ability is enhanced (WO9708333, U.S. Pat. No. 6,319,696), and the like may be used. L-tryptophan-producing bacteria belonging to the genus Escherichia with an enhanced activity of the protein encoded by the yedA gene or the yddG gene may also be used (U.S. patent applications 2003/0148473 A1 and 2003/0157667 A1).

Examples of parent strains for deriving the L-tryptophan-producing bacteria of the present invention also include strains in which one or more activities of the enzymes selected from anthranilate synthase, phosphoglycerate dehydrogenase, and tryptophan synthase are enhanced. The anthranilate synthase and phosphoglycerate dehydrogenase are both subject to feedback inhibition by L-tryptophan and L-serine, so that a mutation desensitizing the feedback inhibition may be introduced into these enzymes. Specific examples of strains having such a mutation include a E. coli SV164 which harbors desensitized anthranilate synthase and a transformant strain obtained by introducing into the E. coli SV164 the plasmid pGH5 (WO 94/08031), which contains a mutant serA gene encoding feedback-desensitized phosphoglycerate dehydrogenase.

Examples of parent strains for deriving the L-tryptophan-producing bacteria of the present invention also include strains transformed with the tryptophan operon which contains a gene encoding desensitized anthranilate synthase (JP 57-71397 A, JP 62-244382 A, U.S. Pat. No. 4,371,614). Moreover, L-tryptophan-producing ability may be imparted by enhancing expression of a gene which encodes tryptophan synthase, among tryptophan operons (trpBA). The tryptophan synthase consists of α and β subunits which are encoded by the trpA and trpB genes, respectively. In addition, L-tryptophan-producing ability may be improved by enhancing expression of the isocitrate lyase-malate synthase operon (WO2005/103275).

L-Proline-Producing Bacteria

Examples of parent strains for deriving L-proline-producing bacteria of the present invention include, but are not limited to, strains belonging to the genus Escherichia, such as E. coli 702ilvA (VKPM B-8012) which is deficient in the ilvA gene and is able to produce L-proline (EP 1172433). The bacterium of the present invention may be improved by enhancing the expression of one or more genes involved in L-proline biosynthesis. Examples of such genes for L-proline producing bacteria which are preferred include the proB gene coding for glutamate kinase desensitized to feedback inhibition by L-proline (DE Patent 3127361). In addition, the bacterium of the present invention may be improved by enhancing the expression of one or more genes coding for proteins excreting L-amino acid from bacterial cell. Such genes are exemplified by b2682 and b2683 genes (ygaZH genes) (EP1239041 A2).

Examples of bacteria belonging to the genus Escherichia, which have an activity to produce L-proline include the following E. coli strains: NRRL B-12403 and NRRL B-12404 (GB Patent 2075056), VKPM B-8012 (Russian patent application 2000124295), plasmid mutants described in DE Patent 3127361, plasmid mutants described by Bloom F. R. et al (The 15^{th} Miami winter symposium, 1983, p. 34), and the like.

L-Arginine-Producing Bacteria

Examples of parent strains for deriving L-arginine-producing bacteria of the present invention include, but are not limited to, strains belonging to the genus Escherichia, such as E. coli strain 237 (VKPM B-7925) (U.S. Patent Application 2002/058315 A1) and its derivative strains harboring mutant N-acetylglutamate synthase (Russian Patent Application No. 2001112869), E. coli strain 382 (VKPM B-7926) (EP1170358A1), an arginine-producing strain into which argA gene encoding N-acetylglutamate synthetase is introduced therein (EP1170361A1), and the like.

Examples of parent strains for deriving L-arginine producing bacteria of the present invention also include strains in which expression of one or more genes encoding an L-arginine biosynthetic enzyme are enhanced. Examples of such genes include genes encoding N-acetylglutamyl phosphate reductase (argC), ornithine acetyl transferase (argJ), N-acetylglutamate kinase (argB), acetylornithine transaminase (argD), ornithine carbamoyl transferase (argF), argininosuccinic acid synthetase (argG), argininosuccinic acid lyase (argH), and carbamoyl phosphate synthetase (carAB).

L-Valine-Producing Bacteria

Example of parent strains for deriving L-valine-producing bacteria of the present invention include, but are not limited to, strains which have been modified to overexpress the ilvGMEDA operon (U.S. Pat. No. 5,998,178). It is desirable to remove the region of the ilvGMEDA operon which is required for attenuation so that expression of the operon is not attenuated by the produced L-valine. Furthermore, the ilvA gene in the operon is desirably disrupted so that threonine deaminase activity is decreased.

Examples of parent strains for deriving L-valine-producing bacteria of the present invention include also include mutants having a mutation of amino-acyl t-RNA synthetase (U.S. Pat. No. 5,658,766). For example, E. coli VL1970, which has a mutation in the ileS gene encoding isoleucine tRNA synthetase, can be used. E. coli VL1970 has been deposited in the Russian National Collection of Industrial Microorganisms (VKPM) (Russia, 113545 Moscow, 1 Dorozhny Proezd, 1) on Jun. 24, 1988 under accession number VKPM B-4411.

Furthermore, mutants requiring lipoic acid for growth and/or lacking H⁺-ATPase can also be used as parent strains (WO96/06926).

L-Isoleucine-Producing Bacteria

Examples of parent strains for deriving L-isoleucine producing bacteria of the present invention include, but are not limited to, mutants having resistance to 6-dimethylaminopurine (JP 5-304969 A), mutants having resistance to an isoleucine analogue such as thiaisoleucine and isoleucine hydroxamate, and mutants additionally having resistance to DL-ethionine and/or arginine hydroxamate (JP 5-130882 A). In addition, recombinant strains transformed with genes encoding proteins involved in L-isoleucine biosynthesis, such as threonine deaminase and acetohydroxate synthase, can also be used as parent strains (JP 2-458 A, FR 0356739, and U.S. Pat. No. 5,998,178).

### 2. Method of the Present Invention

The method of the present invention is a method for producing an L-amino acid by cultivating the bacterium of the present invention in a culture medium to produce and excrete the L-amino acid into the medium, and collecting the L-amino acid from the medium.

In the present invention, the cultivation, collection, and purification of an L-amino acid from the medium and the like may be performed in a manner similar to conventional fermentation methods wherein an amino acid is produced using a bacterium.

The medium used for culture may be either a synthetic or natural medium, so long as it includes a carbon source and a nitrogen source and minerals and, if necessary, appropriate amounts of nutrients which the bacterium requires for growth. The carbon source may include various carbohydrates such as glucose and sucrose, and various organic acids. Depending on the mode of assimilation of the chosen microorganism, alcohol, including ethanol and glycerol, may be used. As the nitrogen source, various ammonium salts such as ammonia and ammonium sulfate, other nitrogen compounds such as amines, a natural nitrogen source such as peptone, soybean-hydrolysate, and digested fermentative microorganism can be used. As minerals, potassium monophosphate, magnesium sulfate, sodium chloride, ferrous sulfate, manganese sulfate, calcium chloride, and the like can be used. As vitamins, thiamine, yeast extract, and the like, can be used.

The cultivation is preferably performed under aerobic conditions, such as a shaking culture, and a stirring culture with aeration, at a temperature of 20 to 40° C., preferably 30 to 38° C. The pH of the culture is usually between 5 and 9, preferably between 6.5 and 7.2. The pH of the culture can be adjusted with ammonia, calcium carbonate, various acids, various bases, and buffers. Usually, a 1 to 5-day cultivation leads to accumulation of the target L-amino acid in the liquid medium.

After cultivation, solids such as cells can be removed from the liquid medium by centrifugation or membrane filtration, and then the L-amino acid can be collected and purified by ion-exchange, concentration, and/or crystallization methods.

### EXAMPLES

The present invention will be more concretely explained below with reference to the following non-limiting Examples.

### Example 1

### Preparation of the PCR Template and Helper Plasmids

The PCR template plasmid pMW118-attL-Cm-attR and the helper plasmid pMW-intxis-ts were prepared as follows:

(1) pMW118-attL-Cm-attR

The pMW118-attL-Cm-attR plasmid was constructed on the basis of pMW118-attL-Tc-attR that was obtained by ligation of the following four DNA fragments:
1) the BglII-EcoRI fragment (114 bp) carrying attL (SEQ ID NO: 11) which was obtained by PCR amplification of the corresponding region of the E. coli W3350 (contained λ prophage) chromosome using oligonucleotides P1 and P2 (SEQ ID NOS: 12 and 13) as primers (these primers contained the subsidiary recognition sites for BglII and EcoRI endonucleases);
2) the PstI-HindIII fragment (182 bp) carrying attR (SEQ ID NO: 14) which was obtained by PCR amplification of the corresponding region of the E. coli W3350 (contained λ prophage) chromosome using the oligonucleotides P3 and P4 (SEQ ID NOS: 15 and 16) as primers (these primers contained the subsidiary recognition sites for PstI and HindIII endonucleases);
3) the large BglII-HindIII fragment (3916 bp) of pMW18-ter_rrnB. The plasmid pMW118-ter_rrnB was obtained by ligation of the following three DNA fragments:
   the large DNA fragment (2359 bp) carrying the AatII-EcoRI fragment of pMW118 that was obtained by the following way: pMW118 was digested with EcoRI restriction endonuclease, treated with Klenow fragment of DNA polymerase I, and then digested with AatII restriction endonuclease;
   the small AatII-BglII fragment (1194 bp) of pUC19 carrying the bla gene for ampicillin resistance (Ap^{R}) was obtained by PCR amplification of the corresponding region of the pUC19 plasmid using oligonucleotides P5 and P6 (SEQ ID NOS: 17 and 18) as primers (these primers contained the subsidiary recognition sites for AatII and BglII endonucleases);
   the small BglII-PstI fragment (363 bp) of the transcription terminator ter_rrnB was obtained by PCR amplification of the corresponding region of the E. coli MG1655 chromosome using the oligonucleotides P7 and P8 (SEQ ID NOS: 19 and 20) as primers (these primers contained the subsidiary recognition sites for BglII and PstI endonucleases);
4) the small EcoRI-PstI fragment (1388 bp) (SEQ ID NO: 21) of pML-Tc-ter_thrL bearing the tetracycline resistance gene and the ter_thrL transcription terminator; the pML-Tc-ter_thrL plasmid was obtained in two steps:
   the pML-ter-thrL plasmid was obtained by digesting the pML-MCS plasmid (Mashko, S. V. et al., Biotekhnologiya (in Russian), 2001, no. 5, 3-20) with the XbaI and BamHI restriction endonucleases followed by ligation of the large fragment (3342 bp) with the XbaI-BamHI fragment (68 bp) carrying terminator ter_thrL obtained by PCR amplification of the corresponding region of the E. coli MG1655 chromosome using oligonucleotides P9 and P10 (SEQ ID NOS: 22 and 23) as primers (these primers contained the subsidiary recognition sites for the XbaI and BamHI endonucleases);
   the pML-Tc-ter_thrL plasmid was obtained by digesting the pML-ter_thrL plasmid with the KpnI and XbaI restriction endonucleases followed by treatment with Klenow fragment of DNA polymerase I and ligation with the small EcoRI-Van91I fragment (1317 bp) of pBR322 bearing the tetracycline resistance gene (pBR322 was digested with EcoRI and Van91I restriction endonucleases and then treated with Klenow fragment of DNA polymerase I);

The above strain E. coli W3350 is a derivative of wild type strain E. coli K-12. The strain E. coli MG1655 (ATCC 700926) is a wild type strain and can be obtained from American Type Culture Collection (P.O. Box 1549 Manassas, Va. 20108, United States of America). The plasmid pMW118 and pUC19 are commercially available. The BglII-EcoRI fragment carrying attL and the BglII-PstI fragment of the transcription terminator ter_rrnB can be obtained from the other strain of E. coli in the same manner as describe above.

The pMW118-attL-Cm-attR plasmid was constructed by ligation of the large BamHI-XbaI fragment (4413 bp) of pMW118-attL-Tc-attR and the artificial DNA BglII-XbaI fragment (1162 bp) containing the P_{A2} promoter (the early promoter of the phage T7), the cat gene for chloramphenicol resistance (Cm^{R}), the ter_thrL transcription terminator, and attR. The artificial DNA fragment (SEQ ID NO: 24) was obtained by the following way:
1. The pML-MCS plasmid was digested with the KpnI and XbaI restriction endonucleases and ligated with the small KpnI-XbaI fragment (120 bp), which included the P_{A2} promoter (the early promoter of phage T7) obtained by PCR amplification of the corresponding DNA region of phage T7 using oligonucleotides P11 and P12 (SEQ ID NOS: 25 and 26, respectively) as primers (these primers contained the subsidiary recognition sites for KpnI and XbaI endonucleases). As a result, the pML-P_{A2}-MCS plasmid was obtained. Complete nucleotide sequence of phage T7 has been reported (J. Mol. Biol., 166: 477-535 (1983).
2. The XbaI site was deleted from pML-P_{A2}-MCS. As a result, the pML-P_{A2}-MCS(XbaI⁻) plasmid was obtained.
3. The small BglII-HindIII fragment (928 bp) of pML-P_{A2}-MCS(XbaI⁻) containing the P_{A2} promoter (the early promoter of the phage T7) and the cat gene for chloramphenicol resistance (Cm^{R}) was ligated with the small HindIII-HindIII fragment (234 bp) of pMW118-attL-Tc-attR containing the ter_thrL transcription terminator and attR.
4. The required artificial DNA fragment (1156 bp) was obtained by PCR amplification of the ligation reaction mixture using oligonucleotides P9 and P4 (SEQ ID NOS: 22 and 16) as primers (these primers contained the subsidiary recognition sites for HindIII and XbaI endonucleases).

(2) pMW-intxis-ts

Recombinant plasmid pMW-intxis-ts containing the cI repressor gene and the int-xis genes of phage λ under control of promoter P_{R} was constructed on the basis of vector pMWP_{lac}lacI-ts. To construct the pMWP_{lac}lacI-ts variant, the AatII-EcoRV fragment of the pMWP_{lac}lacI plasmid (Skorokhodova, A. Yu. et al., Biotekhnologiya (in Russian), 2004, no. 5, 3-21) was substituted with the AatII-EcoRV fragment of the pMAN997 plasmid (Tanaka, K. et al., J. Bacteriol., 2001, 183(22): 6538-6542, WO99/03988) bearing the par and ori loci and the repA^{ts} gene (a temperature sensitive-replication origin) of the pSC101 replicon. The plasmid pMAN997 was constructed by exchanging the VspI-HindIII fragments of pMAN031 (J. Bacteriol., 162, 1196 (1985)) and pUC19.

Two DNA fragments were amplified using phage λ DNA (“Fermentas”) as a template. The first one contained the DNA sequence from 37168 to 38046, the cI repressor gene, promoters P_{RM} and P_{R}, and the leader sequence of the cro gene. This fragment was PCR-amplified using oligonucleotides P13 and P14 (SEQ ID NOS: 27 and 28) as primers. The second DNA fragment containing the xis-int genes of phage λ and the DNA sequence from 27801 to 29100 was PCR-amplified using oligonucleotides P15 and P16 (SEQ ID NOS: 29 and 30) as primers. All primers contained the corresponding restriction sites.

The first PCR-amplified fragment carrying the cI repressor was digested with restriction endonuclease ClaI, treated with Klenow fragment of DNA polymerase I, and then digested with restriction endonuclease EcoRI. The second PCR-amplified fragment was digested with restriction endonucleases EcoRI and PstI. The pMWP_{lac}lacI-ts plasmid was digested with the BglII endonuclease, treated with Klenow fragment of DNA polymerase I, and digested with the PstI restriction endonuclease. The vector fragment of pMWPlaclacI-ts was eluted from agarose gel and ligated with the above-mentioned digested PCR-amplified fragments to obtain the pMW-intxis-ts recombinant plasmid.

### Example 2

### Replacement of the Native Promoter Region for the fucPIKUR Operon in E. coli with the Hybrid P_{L-tac} Promoter

To replace the native promoter region of the fucPICUR operon with a more potent promoter, the DNA fragment carrying the hybrid P_{L-tac} promoter and the chloramphenicol resistance marker (Cm^{R}) encoded by the cat gene was integrated into the chromosome of E. coli MG1655 (ATCC 700926) instead of the native promoter region by the method described by Datsenko K. A. and Wanner B. L. (Proc. Natl. Acad. Sci. USA, 2000, 97: 6640-6645) called “Red-mediated integration” and/or “Red-driven integration”. The pKD46 recombinant plasmid (Datsenko, K. A. and Wanner, B. L., Proc. Natl. Acad. Sci. USA, 2000, 97: 6640-6645) with the thermosensitive replicon was used as a donor of the phage λ-derived genes responsible for the Red-mediated recombination system. The E. coli BW25113 containing the pKD46 recombinant plasmid can be obtained from the E. coli Genetic Stock Center, Yale University, New Haven, U.S.A. (accession number CGSC7630).

The hybrid P_{L-tac} promoter was synthesized chemically (SEQ ID NO: 31). The synthesized DNA fragment containing the hybrid P_{L-tac} promoter bears the BglII recognition site at the 5′ end, which is necessary to further join the cat gene, and a 36-bp region complementary to the 5′ end of the fucPIKUR operon required for further integration into the bacterial chromosome.

The DNA fragment containing the Cm^{R} marker encoded by the cat gene was obtained by PCR, using primers P17 (SEQ ID NO: 32) and P18 (SEQ ID NO: 33) and plasmid pMW118-attL-Cm-attR as a template (for construction see Example 1). Primer P17 contains a 36-bp region complementary to the DNA region located 190 bp upstream of the start codon of the fucPIKUR operon. Primer P18 contains the BglII recognition site at the 5′ end required for ligation to the hybrid P_{L-tac} promoter.

PCR was conducted using a ThermoHybaid PCR Express amplificator (Thermo Electron Corporation). The reaction mixture (in total volume of 50 μl) included 5 μl of PCR buffer (tenfold) containing 15 mM MgCl₂ (“Fermentas”, Lithuania), 200 μM each of dNTP, 25 pmol each of the exploited primers, and 1 U of Taq-polymerase (“Fermentas”, Lithuania). Approximately 5 ng of the plasmid DNA was added to the reaction mixture as a template. The following temperature profile was used: the initial DNA denaturation at 95° C. for 5 min followed by 25 cycles (denaturation at 95° C. for 30 s, annealing at 55° C. for 30 s, elongation at 72° C. for 30 s) and the final elongation at 72° C. for 7 min. Then the amplified DNA fragment was purified by electrophoresis in agarose gel, extracted using “GenElute Spin Columns” (“Sigma”, USA), and precipitated by ethanol.

The DNA fragment containing the hybrid P_{L-tac} promoter and the DNA fragment containing the Cm^{R} marker were treated with BglII and ligated. The ligation product was amplified by PCR using primers P17 (SEQ ID NO: 32) and P19 (SEQ ID NO: 34). Primer P19 contains a 36-bp region at the 5′ end that is complementary to the 5′ end of the fucPIKUR operon and is required for further integration into the bacterial chromosome.

The amplified DNA fragment was purified by electrophoresis in agarose gel, extracted using “GenElute Spin Columns” (“Sigma”, USA), and precipitated by ethanol. The obtained DNA fragment was used for electroporation and Red-mediated integration into the E. coli MG1655/pKD46 chromosome.

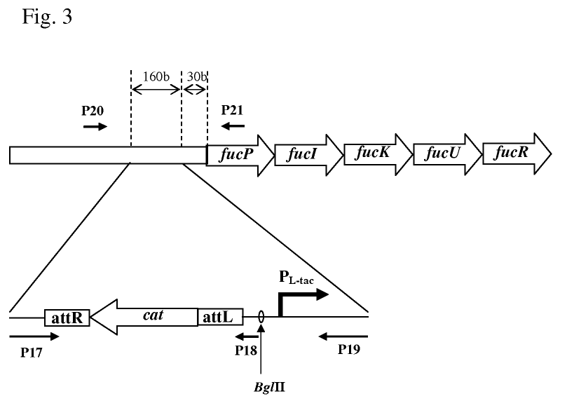

The E. coli MG1655/pKD46 was grown at 30° C. overnight in liquid LB medium supplemented with ampicillin (100 μg/ml). Then, the culture was diluted 100-fold with SOB medium containing yeast extract (5 μl), NaCl (0.5 μl), tryptone (20 μl), KCl (2.5 mM), MgCl₂ (10 mM) supplemented with ampicillin (100 μg/ml) and L-arabinose (10 mM) [arabinose was used to induce the plasmid encoding the Red system genes] and was grown at 30° C. to reach an optical density of OD₆₀₀=0.4-0.7. The cells collected from 10 ml of the bacterial culture were washed three times with ice-cold deionized water and then suspended in 100 μl of the water. The DNA fragment (10 μl, 100 ng) dissolved in deionized water was added to the cell suspension. The electroporation was done using a “BioRad” electroporator (USA, No. 165-2098, version 2-89) according to the manufacturer's instructions. The shocked cells were diluted with 1 ml of SOC medium (Sambrook et al, “Molecular Cloning. A Laboratory Manual, Second Edition”, Cold Spring Harbor Laboratory Press, 1989), incubated at 37° C. for 2 h, and then spread on L-agar containing chloramphenicol (25 μg/ml). After 24 h of growth, colonies were tested for the presence of Cm^{R} marker by PCR using primers P20 (SEQ ID NO: 35) and P21 (SEQ ID NO: 36). For this purpose, a freshly isolated colony was suspended in water (20 μl) and 1 μl of the obtained suspension was used for PCR. The temperature profile was as follows: the initial DNA denaturation at 95° C. for 10 min; then 30 cycles (denaturation at 95° C. for 30 s, annealing at 55° C. for 30 s, and elongation at 72° C. for 1 min), and a final elongation at 72° C. for 7 min. A few Cm^{R} colonies tested contained the required 2000-bp DNA fragment, confirming the substitution of the 190-bp native promoter region of the fucPIKUR operon by the hybrid P_{L-tac} promoter (see FIG. 3). One of the obtained strains was cured from the thermosensitive plasmid pKD46 by culturing at 37° C. and the resulting strain was named E. coli MG1655P_{L-tac}fuCPIKUR.

### Example 3

### Effect of Enhanced Expression of the fucPIKUR Operon on Growth of the E. coli Strain with a Disrupted PTS Transport System

To demonstrate the effect of enhanced expression of the fucPIKUR operon on cell growth, the E. coli strain with a disrupted PTS (phosphoenolpyruvate-sugar transport system) was constructed.

The DNA fragment carrying the kanamycin resistance marker (Km^{R}) was integrated into the chromosome of E. coli MG1655/pKD46 instead of the ptsHI-crr operon by the method described by Datsenko K. A. and Wanner B. L. (Proc. Natl. Acad. Sci. USA, 2000, 97, 6640-6645) called “Red-mediated integration” and/or “Red-driven integration” as described in Example 2.

The ptsHI-crr operon has been elucidated (nucleotide positions: 2531786 to 2532043, 2532088 to 2533815, and 2533856 to 2534365 for ptsH, ptsI, and crr genes, respectively; GenBank accession no. NC_{—}000913.2; gi: 49175990). The ptsHI-crr operon is located between cysK and pdxK genes on the E. coli K-12 chromosome.

The DNA fragment carrying the Km^{R} gene was obtained by PCR using the commercially available plasmid pUC4KAN (GenBank/EMBL accession no. X06404; “Fermentas”, Lithuania) as the template and primers P22 (SEQ ID NO: 37) and P23 (SEQ ID NO: 38). Primer P22 contained a 36-nt sequence complementary to the 5′ end of the ptsH gene and primer P23 contained a 36-nt sequence complementary to the 3′ end of the crr gene. These nucleotide sequences were introduced into primers P22 and P23 for further integration into the bacterial chromosome.

PCR was conducted as described in Example 2.

The PCR-amplified DNA fragment was purified by agarose gel-electrophoresis, extracted from the gel by centrifugation through a “GenElute Spin Column” (“Sigma”, USA), and precipitated by ethanol. The obtained DNA fragment was used for electroporation and Red-mediated integration into the chromosome of E. coli MG1655/pKD46 as described in Example 2, except that after electroporation, cells were spread onto L-agar containing 50 μg/ml of kanamycin.

After 24 h of growth, the bacterial colonies were tested for the presence of the Km^{R} marker instead of ptsHI-crr operon by PCR using primers P24 (SEQ ID NO: 39) and P25 (SEQ ID NO: 40). For this purpose, a freshly isolated colony was suspended in 20 μl of water, and 1 μl of the resulting suspension was used for PCR. The PCR conditions were described in Example 2. A few Km^{R} colonies tested contained the required 1300 bp DNA fragment, which confirmed the presence of the Km^{R} gene instead of the ptsHI-crr operon. One of the strains obtained was cured from thermosensitive plasmid pKD46 by culturing at 37° C., and the resulting strain was named E. coli MG1655 ΔptsHI-crr.

The DNA fragment from the chromosome of the above-mentioned E. coli MG1655P_{L-tac}fucPIKUR was transferred to E. coli MG1655 ΔptsHI-crr by P1 transduction (Miller, J. H. (1972) Experiments in Molecular Genetics, Cold Spring Harbor Lab. Press, Plainview, N.Y.), resulting in strain MG1655 ΔptsHI-crr P_{L-tac}fucPIKUR.

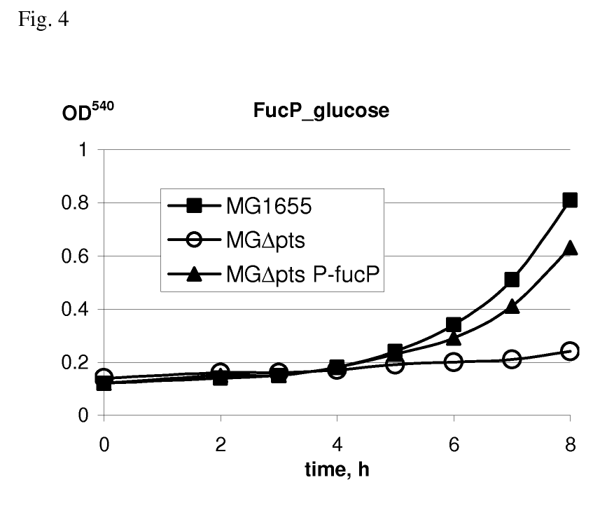

The ability to grow on minimal medium with glucose (4%) as a carbon source was verified for the following three E. coli strains: MG1655, MG1655 ΔptsHI-crr, and MG1655 ΔptsHI_crr P_{L-tac}fucPIKUR (FIG. 4). As follows from FIG. 4, E. coli MG1655 ΔptsHI-crr P_{L-tac}fucPIKUR demonstrated a substantially higher growth rate, as compared with MG1655 ΔptsHI-crr, indicating the fact that enhancing expression of the fucPIKUR operon significantly increased the growth characteristics of the recipient strain on minimal medium containing glucose.

### Example 4

### Production of L-Threonine by E. coli B-3996P_{L-tac}fucPIKUR

To test the effect of enhanced expression of the fucPIKUR operon (under the P_{L-tac} promoter control) on threonine production, DNA fragments from the chromosome of the above-described E. coli strain MG1655P_{L-tac}fucPIKUR were transferred to the threonine-producing E. coli strain B-3996 (VKPM B-3996) by P1 transduction (Miller, J. H. Experiments in Molecular Genetics, Cold Spring Harbor Lab. Press, 1972, Plainview, N.Y.).

Both E. coli strains, B-3996 and B-3996P_{L-tac}fucPIKUR, were grown for 18-24 hours at 37° C. on L-agar plates. To obtain a seed culture, the strains were grown on a rotary shaker (250 rpm) at 32° C. for 18 hours in 20×200-mm test tubes containing 2 ml of L-broth supplemented with 4% glucose. Then, the fermentation medium was inoculated with 0.21 ml (10%) seed material. The fermentation was performed in 2 ml of minimal medium for fermentation in 20×200-mm test tubes. Cells were grown for 72 hours at 32° C. with shaking at 250 rpm.

After cultivation, the amount of L-threonine, which had accumulated in the medium, was determined by paper chromatography using the following mobile phase: butanol-acetic acid-water, 4:1:1 (v/v). A solution of ninhydrin (2%) in acetone was used as a visualizing reagent. A spot containing L-threonine was cut out, L-threonine was eluted with 0.5% water solution of CdCl₂, and the amount of L-threonine was estimated spectrophotometrically at 540 nm. The results of four independent test tube fermentations are shown in Table 1.

The composition of the fermentation medium (g/l) was as follows:

| | |
|---|---|
| Glucose | 80.0 |
| (NH₄)₂SO₄ | 22.0 |
| NaCl | 0.8 |
| KH₂PO₄ | 2.0 |
| MgSO₄•7H₂O | 0.8 |
| FeSO₄•7H₂O | 0.02 |
| MnSO₄•5H₂O | 0.02 |
| Thiamine HCl | 0.0002 |
| Yeast extract | 1.0 |
| CaCO₃ | 30.0 |

Glucose and magnesium sulfate were sterilized separately. CaCO₃ was sterilized by dry-heat at 180° C. for 2 hours. The pH was adjusted to 7.0. The antibiotic was added to the medium after sterilization.

TABLE 1

| | 48 h | | | |
|---|---|---|---|---|
| | | L- | 72 h | |
| | | threonine, | | L-threonine, |
| Strain | OD₅₄₀ | g/l | OD₅₄₀ | g/l |
| B-3996 | 19.1 ± 0.3 | 16.2 ± 0.5 | 17.1 ± 0.3 | 24.8 ± 0.3 |
| B-3996P_{L-tac}fucPIKUR | 18.9 ± 0.2 | 18.0 ± 0.4 | 17.9 ± 0.3 | 29.2 ± 0.6 |

As follows from Table 1, B-3996P_{L-tac}fucPIKUR produced a higher amount of L-threonine, as compared with B-3996.

### Example 5

### Production of L-Lysine by E. coli WC196(pCABD2)P_{L-tac}fucPIKUR

To test the effect of enhanced expression of the fucPIKUR operon (under the P_{L-tuc} promoter control) on lysine production, DNA fragments from the chromosome of the above-described E. coli strain MG1655P_{L-tac}fucPIKUR can be transferred to the lysine-producing E. coli strain WC196 (pCABD2) by P1 transduction (Miller, J. H. Experiments in Molecular Genetics, Cold Spring Harbor Lab. Press, 1972, Plainview, N.Y.). The pCABD2 plasmid includes the dapA gene encoding dihydrodipicolinate synthase having a mutation which desensitizes the feedback inhibition by L-lysine, the lysC gene encoding aspartokinase III having a mutation which desensitizes the feedback inhibition by L-lysine, the dapB gene encoding dihydrodipicolinate reductase, and the ddh gene encoding diaminopimelate dehydrogenase (U.S. Pat. No. 6,040,160).

Both E. coli strains, WC196(pCABD2) and WC196(pCABD2)P_{L-tac}fucPIKUR, can be cultured in L-medium containing streptomycin (20 mg/l) at 37° C., and 0.3 ml of the obtained culture can be inoculated into 20 ml of the fermentation medium containing the required drugs in a 500-ml flask. The cultivation can be carried out at 37° C. for 16 h by using a reciprocal shaker at the agitation speed of 115 rpm. After the cultivation, the amounts of L-lysine and residual glucose in the medium can be measured by a known method (Biotech-analyzer AS210 manufactured by Sakura Seiki Co.). Then, the yield of L-lysine can be calculated based on consumed glucose for each of the strains.

The composition of fermentation medium (g/l) is as follows:

| | |
|---|---|
| Glucose | 40 |
| (NH₄)₂SO₄ | 24 |
| K₂HPO₄ | 1.0 |
| MgSO₄•7H₂O | 1.0 |
| FeSO₄•7H₂O | 0.01 |
| MnSO₄•5H₂O | 0.01 |
| Yeast extract | 2.0 |

The pH is adjusted to 7.0 by KOH and the medium is autoclaved at 115° C. for 10 min. Glucose and MgSO₄.7H₂O are sterilized separately. CaCO₃ is dry-heat sterilized at 180° C. for 2 hours and added to the medium for a final concentration of 30 μl.

### Example 6

### Production of L-Cysteine by E. coli JM15(ydeD)P_{L-tac}fucPIKUR

To test the effect of enhanced expression of the fucPIKUR operon (under the P_{L-tac} promoter control) on L-cysteine production, DNA fragments from the chromosome of the above-described E. coli strain MG1655P_{L-tac}fucPIKUR can be transferred to the L-cysteine-producing E. coli strain JM15(ydeD) by P1 transduction (Miller, J. H. Experiments in Molecular Genetics, Cold Spring Harbor Lab. Press, 1972, Plainview, N.Y.) to obtain the strain JM15(ydeD)P_{L-tac}fucPIKUR.

E. coli JM15(ydeD) is a derivative of E. coli JM15 (U.S. Pat. No. 6,218,168), which can be transformed with DNA having the ydeD gene encoding a membrane protein, and is not involved in a biosynthetic pathway of any L-amino acid (U.S. Pat. No. 5,972,663). The strain JM15 (CGSC #5042) can be obtained from The Coli Genetic Stock Collection at the E. coli Genetic Resource Center, MCD Biology Department, Yale University (http://cgsc.biology.yale.edu/).

Fermentation conditions for evaluation of L-cysteine production were described in detail in Example 6 of U.S. Pat. No. 6,218,168.

### Example 7

### Production of L-Leucine by E. coli 57P_{L-tac}fucPIKUR

To test the effect of enhanced expression of the fucPIKUR operon (under the P_{L-tac} promoter control) on L-leucine production, DNA fragments from the chromosome of the above-described E. coli strain MG1655P_{L-tac}fucPIKUR can be transferred to the L-leucine-producing E. coli strain 57 (VKPM B-7386, U.S. Pat. No. 6,124,121) by P1 transduction (Miller, J. H. Experiments in Molecular Genetics, Cold Spring Harbor Lab. Press, 1972, Plainview, N.Y.) to obtain the strain 57-pMW-ΔfucPIKUR. The strain 57 has been deposited in the Russian National Collection of Industrial Microorganisms (VKPM) (Russia, 117545 Moscow, 1 Dorozhny proezd, 1) on May 19, 1997 under accession number VKPM B-7386.

Both E. coli strains, 57 and 57P_{L-tac}fucPIKUR, can be cultured for 18-24 hours at 37° C. on L-agar plates. To obtain a seed culture, the strains can be grown on a rotary shaker (250 rpm) at 32° C. for 18 hours in 20×200-mm test tubes containing 2 ml of L-broth supplemented with 4% glucose. Then, the fermentation medium can be inoculated with 0.21 ml of seed material (10%). The fermentation can be performed in 2 ml of a minimal fermentation medium in 20×200-mm test tubes. Cells can be grown for 48-72 hours at 32° C. with shaking at 250 rpm. The amount of L-leucine can be measured by paper chromatography (liquid phase composition: butanol-acetic acid-water=4:1:1)

The composition of the fermentation medium (g/l) is as follows (pH 7.2):

| | |
|---|---|
| Glucose | 60.0 |
| (NH₄)₂SO₄ | 25.0 |
| K₂HPO₄ | 2.0 |
| MgSO₄•7H₂O | 1.0 |
| Thiamine | 0.01 |
| CaCO₃ | 25.0 |

Glucose and CaCO₃ are sterilized separately.

### Example 8

### Production of L-Histidine by E. coli 80P_{L-tac}fucPIKUR

To test the effect of enhanced expression of the fucPIKUR operon (under the P_{L-tac} promoter control) on L-histidine production, DNA fragments from the chromosome of the above-described E. coli strain MG1655P_{L-tac}fucPIKUR can be transferred to the histidine-producing E. coli strain 80 by P1 transduction (Miller, J. H. Experiments in Molecular Genetics, Cold Spring Harbor Lab. Press, 1972, Plainview, N.Y.). The strain 80 has been described in Russian patent 2119536 and deposited in the Russian National Collection of Industrial Microorganisms (Russia, 117545 Moscow, 1 Dorozhny proezd, 1) on Oct. 15, 1999 under accession number VKPM B-7270 and then converted to a deposit under the Budapest Treaty on Jul. 12, 2004.

Both E. coli strains, 80 and 80P_{L-tac}fucPIKUR, can be cultured in L-broth for 6 hours at 29° C. Then, 0.1 ml of obtained culture can be inoculated into 2 ml of fermentation medium in a 20×200-mm test tube and cultivated for 65 hours at 29° C. with shaking on a rotary shaker (350 rpm). After cultivation, the amount of histidine which accumulates in the medium can be determined by paper chromatography. The paper can be developed with a mobile phase consisting of n-butanol:acetic acid:water=4:1:1 (v/v). A solution of ninhydrin (0.5%) in acetone can be used as a visualizing reagent.

The composition of the fermentation medium (pH 6.0) is as follows (g/l):

| | |
|---|---|
| Glucose | 100.0 |
| Mameno (soybean hydrolysate) | 0.2 as total nitrogen |
| L-proline | 1.0 |
| (NH₄)₂SO₄ | 25.0 |
| KH₂PO₄ | 2.0 |
| MgSO₄•7H₂0 | 1.0 |
| FeSO₄•7H₂0 | 0.01 |
| MnSO₄ | 0.01 |
| Thiamine | 0.001 |
| Betaine | 2.0 |
| CaCO₃ | 60.0 |

Glucose, proline, betaine and CaCO₃ are sterilized separately. The pH is adjusted to 6.0 before sterilization.

### Example 9

### Production of L-Glutamate by E. coli VL334thrC⁺P_{L-tac}fucPIKUR

To test the effect of enhanced expression of the fucPIKUR operon (under the P_{L-tac} promoter control) on L-glutamate production, DNA fragments from the chromosome of the above-described E. coli strain MG1655P_{L-tac}fucPIKUR can be transferred to the L-glutamate-producing E. coli strain VL334thrC⁺ (EP 1172433) by P1 transduction (Miller, J. H. Experiments in Molecular Genetics, Cold Spring Harbor Lab. Press, 1972, Plainview, N.Y.) to obtain the strain VL334thrC⁺P_{L-tac}fucPIKUR. The strain VL334thrC⁺ has been deposited in the Russian National Collection of Industrial Microorganisms (VKPM) (Russia, 117545 Moscow, 1 Dorozhny proezd, 1) on Dec. 6, 2004 under the accession number VKPM B-8961 and then converted to a deposit under the Budapest Treaty on Dec. 8, 2004.

Both E. coli strains, VL334thrC⁺ and VL334thrC⁺P_{L-tac}fucPIKUR, can be grown for 18-24 hours at 37° C. on L-agar plates. Then, one loop of the cells can be transferred into test tubes containing 2 ml of fermentation medium. The fermentation medium contains glucose (60g/l), ammonium sulfate (25 μl), KH₂PO₄ (2g/l), MgSO₄ (1 μl), thiamine (0.1 mg/ml), L-isoleucine (70 μg/ml), and CaCO₃ (25 μl). The pH is adjusted to 7.2. Glucose and CaCO₃ are sterilized separately. Cultivation can be carried out at 30° C. for 3 days with shaking. After the cultivation, the amount of L-glutamic acid produced can be determined by paper chromatography (liquid phase composition of butanol-acetic acid-water=4:1:1) with subsequent staining by ninhydrin (1% solution in acetone) and further elution of the compounds in 50% ethanol with 0.5% CdCl₂.

### Example 10

### Production of L-Phenylalanine by E. coli AJ12739P_{L-tac}fuCPIKUR

To test the effect of enhanced expression of the fucPIKUR operon (under the P_{L-tuc} promoter control) on L-phenylalanine production, DNA fragments from the chromosome of the above-described E. coli strain MG1655P_{L-tac}fucPIKUR can be transferred to the phenylalanine-producing E. coli strain AJ12739 by P1 transduction (Miller, J. H. Experiments in Molecular Genetics, Cold Spring Harbor Lab. Press, 1972, Plainview, N.Y.). The strain AJ12739 has been deposited in the Russian National Collection of Industrial Microorganisms (VKPM) (Russia, 117545 Moscow, 1 Dorozhny proezd, 1) on Nov. 6, 2001 under accession number VKPM B-8197 and then converted to a deposit under the Budapest Treaty on Aug. 23, 2002.

Both E. coli strains, AJ12739 and AJ12739P_{L-tac}fucPIKUR, can each be cultivated at 37° C. for 18 hours in a nutrient broth, and 0.3 ml of the obtained culture can be inoculated into 3 ml of a fermentation medium in a 20×200-mm test tube and cultivated at 37° C. for 48 hours with shaking on a rotary shaker. After cultivation, the amount of phenylalanine which accumulates in the medium can be determined by TLC. The 10×15-cm TLC plates coated with 0.11-mm layers of Sorbfil silica gel containing no fluorescent indicator (Stock Company Sorbpolymer, Krasnodar, Russia) can be used. The Sorbfil plates can be developed with a mobile phase consisting of propan-2-ol-ethyl acetate: 25% aqueous ammonia:water=40:40:7: 16 (v/v). A solution of ninhydrin (2%) in acetone can be used as a visualizing reagent.

The composition of the fermentation medium (g/l) is as follows:

| | |
|---|---|
| Glucose | 40.0 |
| (NH₄)₂SO₄ | 16.0 |
| K₂HPO₄ | 0.1 |
| MgSO₄•7H₂O | 1.0 |
| FeSO₄•7H₂O | 0.01 |
| MnSO₄•5H₂O | 0.01 |
| Thiamine HCl | 0.0002 |
| Yeast extract | 2.0 |
| Tyrosine | 0.125 |
| CaCO₃ | 20.0 |

Glucose and magnesium sulfate are sterilized separately. CaCO₃ is dry-heat sterilized at 180° for 2 hours. The pH is adjusted to 7.0.

### Example 11

### Production of L-Tryptophan by E. coli SV164 (pGH5)P_{L-tac}fucPIKUR

To test the effect of enhanced expression of the fucPIKUR operon (under the P_{L-tuc} promoter control) on L-tryptophan production, DNA fragments from the chromosome of the above-described E. coli strain MG1655P_{L-tac}fucPIKUR can be transferred to the tryptophan-producing E. coli strain SV164 (pGH5) by P1 transduction (Miller, J. H. Experiments in Molecular Genetics, Cold Spring Harbor Lab. Press, 1972, Plainview, N.Y.). The strain SV164 has the trpE allele encoding anthranilate synthase free from feedback inhibition by tryptophan. The plasmid pGH5 harbors a mutant serA gene encoding phosphoglycerate dehydrogenase not subject to feedback inhibition by serine. The strain SV164 (pGH5) is described in detail in U.S. Pat. No. 6,180,373.

Both E. coli strains, SV164(pGH5) and SV164(pGH5)P_{L-tac}fucPIKUR, can be cultivated with shaking at 37° C. for 18 hours in 3 ml of nutrient broth supplemented with tetracycline (20 mg/l, marker of pGH5 plasmid). The obtained cultures (0.3 ml each) can be each inoculated into 3 ml of a fermentation medium containing tetracycline (20 mg/l) in 20×200-mm test tubes, and cultivated at 37° C. for 48 hours with a rotary shaker at 250 rpm. After cultivation, the amount of tryptophan which accumulates in the medium can be determined by TLC as described in Example 10. The fermentation medium components are listed in Table 2, and are sterilized in separate groups (A, B, C, D, E, F, and H), as shown, to avoid adverse interactions during sterilization.

TABLE 2

| Groups | Component | Final concentration, g/l |
|---|---|---|
| A | KH₂PO₄ | 1.5 |
| | NaCl | 0.5 |
| | (NH₄)₂SO₄ | 1.5 |
| | L-Methionine | 0.05 |
| | L-Phenylalanine | 0.1 |
| | L-Tyrosine | 0.1 |
| | Mameno (total N) | 0.07 |
| B | Glucose | 40.0 |
| | MgSO₄•7H₂O | 0.3 |
| C | CaCl₂ | 0.011 |
| D | FeSO₄•7H₂O | 0.075 |
| | Sodium citrate | 1.0 |
| E | Na₂MoO₄•2H₂O | 0.00015 |
| | H₃BO₃ | 0.0025 |
| | CoCl₂•6H₂O | 0.00007 |
| | CuSO₄•5H₂O | 0.00025 |
| | MnCl₂•4H₂O | 0.0016 |
| | ZnSO₄•7 H₂O | 0.0003 |
| F | Thiamine HCl | 0.005 |
| G | CaCO₃ | 30.0 |
| H | Pyridoxine | 0.03 |

Group A has pH 7.1 adjusted by NH₄OH. Each of groups A, B, C, D, E, F and His sterilized separately, chilled, and mixed together, and then CaCO₃ sterilized by dry heat is added to the complete fermentation medium.

### Example 12

### Production of L-Proline by E. coli 702ilvAP_{L-tac}fuCPIKUR

To test the effect of enhanced expression of the fucPIKUR operon (under the P_{L-tuc} promoter control) on L-proline production, DNA fragments from the chromosome of the above-described E. coli strain MG1655P_{L-tac}fucPIKUR can be transferred to the proline-producing E. coli strain 702ilvA by P1 transduction (Miller, J. H. Experiments in Molecular Genetics, Cold Spring Harbor Lab. Press, 1972, Plainview, N.Y.). The strain 702ilvA has been deposited in the Russian National Collection of Industrial Microorganisms (VKPM) (Russia, 117545 Moscow, 1 Dorozhny proezd, 1) on Jul. 18, 2000 under accession number VKPM B-8012 and then converted to a deposit under the Budapest Treaty on May 18, 2001.

Both E. coli strains, 702ilvA and 702ilvAP_{L-tac}fucPIKUR, can be grown for 18-24 hours at 37° C. on L-agar plates. Then, these strains can be cultivated under the same conditions as in Example 9.

### Example 13

### Production of L-Arginine by E. coli 382P_{L-tac}fucPIKUR

To test the effect of enhanced expression of the fucPIKUR operon (under the P_{L-tac} promoter control) on L-arginine production, DNA fragments from the chromosome of the above-described E. coli strain MG1655P_{L-tac}fucPIKUR can be transferred to the arginine-producing E. coli strain 382 by P1 transduction (Miller, J. H. Experiments in Molecular Genetics, Cold Spring Harbor Lab. Press, 1972, Plainview, N.Y.). The strain 382 has been deposited in the Russian National Collection of Industrial Microorganisms (VKPM) (Russia, 117545 Moscow, 1 Dorozhny proezd, 1) on Apr. 10, 2000 under accession number VKPM B-7926 and then converted to a deposit under the Budapest Treaty on May 18, 2001.

Both strains, 382 and 382P_{L-tac}fucPIKUR, can be cultivated with shaking at 37° C. for 18 hours in 3 ml of nutrient broth. The obtained cultures (0.3 ml each) can be inoculated into 3 ml of a fermentation medium in 20×200-mm test tubes and cultivated at 32° C. for 48 hours on a rotary shaker.

After the cultivation, the amount of L-arginine which accumulates in the medium can be determined by paper chromatography using the following mobile phase:butanol:acetic acid:water=4:1:1 (v/v). A solution of ninhydrin (2%) in acetone can be used as a visualizing reagent. A spot containing L-arginine can be cut out, L-arginine can be eluted with 0.5% water solution of CdCl₂, and the amount of L-arginine can be estimated spectrophotometrically at 540 nm.

The composition of the fermentation medium (g/l) is as follows:

| | |
|---|---|
| Glucose | 48.0 |
| (NH4)₂SO₄ | 35.0 |
| KH₂PO₄ | 2.0 |
| MgSO₄•7H₂O | 1.0 |
| Thiamine HCl | 0.0002 |
| Yeast extract | 1.0 |
| L-isoleucine | 0.1 |
| CaCO3 | 5.0 |

Glucose and magnesium sulfate are sterilized separately. CaCO₃ is dry-heat sterilized at 180° C. for 2 hours. The pH is adjusted to 7.0.

### Example 14

### Elimination of the Cm Resistance Gene (cat Gene) from the Chromosome of L-Amino Acid Producing E. coli Strains.

Cm resistance gene (cat gene) can be eliminated from the chromosome of the L-amino acid producing strain using int-xis system. For that purpose, L-amino acid producing strains, in which DNA fragments from the chromosome of the above-described E. coli strain MG1655P_{L-tac}fucPIKUR was transferred by P1 transduction (see Examples 4-13), can be transformed with plasmid pMWts-Int/X is. Transformant clones can be selected on the LB-medium containing 100 μg/ml of ampicillin. Plates can be incubated overnight at 30° C. Transformant clones can be cured from cat gene by spreading the separate colonies at 37° C. (at that temperature repressor CIts is partially inactivated and transcription of int/xis genes is derepressed) followed by selection of Cm^{S}Ap^{R} variants. Elimination of the cat gene from the chromosome of the strain can be verified by PCR. Locus-specific primers P20 (SEQ ID NO: 35) and P21 (SEQ ID NO: 36) can be used in PCR for the verification. Conditions for PCR verification can be as described above. The PCR product obtained in the reaction with the cells with eliminated cat gene as a template, should be 0.2 kbp in length. Thus, the L-amino acid producing strain with enhanced expression of fucPIKUR operon and eliminated cat gene can be obtained.

While the invention has been described in detail with reference to preferred embodiments thereof, it will be apparent to one skilled in the art that various changes can be made, and equivalents employed, without departing from the scope of the invention. All the cited references herein are incorporated as a part of this application by reference.

### INDUSTRIAL APPLICABILITY

According to the present invention, production of L-amino acid of a bacterium of the Enterobacteriaceae family can be enhanced.

1. An L-amino acid-producing bacterium of the Enterobacteriaceae family, wherein said bacterium has been modified to enhance expression of at least one gene of the fucPIKUR operon.

2. The bacterium according to claim 1, wherein said gene expression is enhanced by modifying an expression control sequence for the fucPIKUR operon.

3. The bacterium according to claim 1, wherein said gene expression is enhanced by increasing the copy number for at least one gene of the fucPIKUR operon.

4. The bacterium according to claim 1, wherein said bacterium belongs to the genus Escherichia.

5. The bacterium according to claim 1, wherein said bacterium belongs to the genus Pantoea.

6. The L-amino acid-producing bacterium according to claim 1, wherein said L-amino acid is selected from the group consisting of an aromatic L-amino acid and a non-aromatic L-amino acid.

7. The L-amino acid-producing bacterium according to claim 6, wherein said aromatic L-amino acid is selected from the group consisting of L-phenylalanine, L-tyrosine, and L-tryptophan.

8. The L-amino acid-producing bacterium according to claim 6, wherein said non-aromatic L-amino acid is selected from the group consisting of L-threonine, L-lysine, L-cysteine, L-methionine, L-leucine, L-isoleucine, L-valine, L-histidine, glycine, L-serine, L-alanine, L-asparagine, L-aspartic acid, L-glutamine, L-glutamic acid, L-proline, and L-arginine.

9. A method for producing an L-amino acid comprising:
cultivating the bacterium according to claim 1 in a medium to produce and excrete said L-amino acid into the medium, and
collecting said L-amino acid from the medium.

10. The method according to claim 9, wherein said L-amino acid is selected from the group consisting of an aromatic L-amino acid and a non-aromatic L-amino acid.

11. The method according to claim 10, wherein said aromatic L-amino acid is selected from the group consisting of L-phenylalanine, L-tyrosine, and L-tryptophan.

12. The method according to claim 10, wherein said non-aromatic L-amino acid is selected from the group consisting of L-threonine, L-lysine, L-cysteine, L-methionine, L-leucine, L-isoleucine, L-valine, L-histidine, L-glycine, L-serine, L-alanine, L-asparagine, L-aspartic acid, L-glutamine, L-glutamic acid, L-proline, and L-arginine.
